(19) United States
(12) Patent Application Publication
(10) Pub. No.: US 2022/0125784 A1
(43) Pub. Date: Apr. 28, 2022

(54) METHODS AND COMPOUNDS FOR INHIBITION OF INACTIVATION OF VOLTAGE-GATED SODIUM CHANNELS

(71) Applicant: SIMON FRASER UNIVERSITY, Burnaby (CA)

(72) Inventors: Mena F. ABDELSAYED, Port Coquitlam (CA); Peter C. RUBEN, Anmore (CA)

(21) Appl. No.: 17/427,949

(22) PCT Filed: Feb. 4, 2020
(86) PCT No.: PCT/IB2020/050853

§ 371 Date: Aug. 3, 2021

Related U.S. Application Data
(60) Provisional application No. 62/800,932, filed on Feb. 4, 2019.

(51) Int. Cl. A61K 31/498; A61K 31/4184

(52) U.S. Cl. CPC A61K 31/498; A61K 31/4184

(57) ABSTRACT

The current application relates to compounds that bind and inhibit the inactivation of Navi.5 voltage-gated sodium channel (VGSC). The compounds can be used for treating cardiovascular diseases such as Brugada syndrome, cardiac arrhythmia disorder, progressive cardiac conduction disorder (PCCD), sick sinus syndrome, progressive familial block, atrial fibrillation, sudden infant death syndrome, dilated cardiomyopathy, myocardial ischemia/infarction, or heart failure.

### FIELD OF INVENTION

The present invention relates to methods for inhibiting the inactivation of a voltage-gated sodium channel and uses thereof. More specifically, the present invention relates to methods for inhibiting the inactivation of a Naᵥ1.5 voltage-gated sodium channel and uses thereof.

### BACKGROUND OF THE INVENTION

Cardiovascular disease (CVD) is a common cause of death world-wide ^{1,2}. If left untreated, CVD can cause cardiac arrest and sudden cardiac death (SCD), leaving behind devastated families as it targets various age-groups: infants during sleep (known as sudden infant death syndrome, SIDS), athletes, and adults. CVD represents a major economic burden on health care systems, especially in urbanized countries. The mechanism underlying SCD is usually an abnormal heart rhythm, known as an arrhythmia, generated by irregularly functioning cardiac proteins. Normal protein function is required for the regulation and spread of the electrical signal, the cardiac action potential, that triggers the heartbeat. Protein function is disrupted by CVD.

The cardiac voltage-gated sodium channel (Naᵥ1.5) is one of a family of proteins, collectively known as ion channels, responsible for electrical excitation in cardiac tissue. The SCN5a gene, located in the short (p) arm of chromosome 3 at position 22.2, encodes the Naᵥ1.5 isoform, expressed predominantly in cardiac myocytes and in Purkinje fibers. The primary sequence of Naᵥ1.5 includes six transmembrane segments found in each of the four domains. The auxiliary β subunit binds to the main subunit of the channel. Various intracellular molecules and proteins, including ankyrin-G, fibroblast growth factor homologous factor 1, multicopy suppressor of Gsp1, neural precursor cell expressed developmentally downregulated protein 4, caveolin-3, nitric oxide synthase, postsynaptic density protein/Drosophila disk large tumor suppressor/zonula occludens-1, α₁-syntrophin, lle-Phe-Met, reactive oxygen species, mitochondria, etc. modify channel gating ¹⁵. The main Naᵥ1.5 α-subunit (alpha-subunit) consists of four domains (Domain I-IV), each with six α-helices (alpha-helices; S1-S6) ¹⁵. The first four helices (S1-S4) are a voltage-sensing segment, which is displaced with depolarization (a positive change in the cellular membrane potential, V_{M}). The displacement imposes a mechanical force on the pore-forming segments (S5-S6), resulting in channel opening ³⁻⁵. Channel activation allows for sodium current (I_{Na}) to move into and further depolarize the cell. Following activation, a small series of hydrophobic amino acid residues (the IFMT motif), found in the Domain III-IV linker, binds to the hydrophobic lip of the pore, causing fast inactivation ₆₋₆. Some channels transition back into the open state giving rise to late sodium current (late I_{Na}) ⁹. Upon maintained depolarization, the channel enters into the slow inactivated state. Slow inactivation is mechanistically and pharmacologically different from fast inactivation ^{10,11}. Slow inactivation occurs over several seconds and is thought to involve conformational changes in the selectivity filter, voltage sensors, and lateral pores known as fenestrations ¹². The membrane potential must be repolarized (restored to its negative resting value) to recover channels from fast and slow inactivation. Auxiliary proteins and molecules modify Naᵥ1.5 expression and gating: the β1 subunit, cytosolic calcium, and phosphorylation proteins including Ca²⁺/calmodulin-dependent protein kinase II, protein kinase A, and protein kinase C¹³⁻¹⁵.

Cardiac disease (both inherited and non-inherited) is thought to be associated with cellular disturbances in cardiomyocytes, leading to the activation of multiple cellular signaling cascades which activate intracellular kinases (phosphorylation proteins) and elevates cytosolic calcium ^{16,17}. Kinase expression levels also increases with cardiac disease ¹⁸. Consequently, Naᵥ1.5 behavior is altered, expressing both gain-of-function and loss-of-function. Increased phosphorylation in Naᵥ1.5 elevates late I_{Na} and enhances entry into fast and slow inactivation ^{15,18,19}. With elevated heart rates, the rapid onset into fast inactivation terminates the peak sodium current and reduces action potential amplitude. Channels also accumulate into slow inactivation with elevated stimulation frequencies, substantially dropping the available Naᵥ1.5 channels, required for heart rhythm maintenance ¹⁵. The loss in Naᵥ1.5 availability underlies arrhythmias. The biophysical defects are often caused by genetic diseases in which mutations in the SCN5a gene perturb normal Naᵥ1.5 function ^{20,21}. For example, multiple Naᵥ1.5 mutants located in the Domain III-IV intracellular linker and the C-terminal regions cause the arrhythmogenic syndrome, Long-QT, which prolongs repolarization in cardiomyocytes by increasing late I_{Na}. These mutants may also give rise to Brugada Syndrome, which augments the ST-elevation in the right precordial leads by enhancing entry into inactivation and/or shifting the voltage-dependence of inactivation to more negative potentials, thereby decreasing channel availability ²². Other cardiac arrhythmia disorders are unmasked by conditions of loss-of-function in Naᵥ1.5: inherited disorders include progressive cardiac conduction disorder (PCCD), sick sinus syndrome, progressive familial heart block, atrial fibrillation, sudden infant death syndrome, dilated cardiomyopathy, and acquired diseases like myocardial ischemia/infarction ^{23,24}.

Fast inactivation is structurally distinct from slow inactivation. Different channel sites rearrange during slow inactivation. Rearrangement in the fenestrations have been implicated as an important pathway for lipophilic drug entry into the channel's pore ²⁵. Structural and electrophysiological studies have shown that bulky compounds, such as flecainide, elicit their state-dependent effects on Naᵥ via the fenestrations 26,27. Comparison between the structural models of the closed and open voltage-gated Na(+) channel from Arcobacter butzleri (NaᵥAb) and Magnetococcus marinus (NaᵥM) showed a state-dependent difference in fenestration size. While the channels were at rest, the fenestrations, which are identical in all four subunits of the NaᵥAb and NaᵥM, are considerably larger during the closed-state as opposed to the open state, especially in NaᵥAb. Resting-state drug block may be accounted for by this mechanism ²⁸.

The voltage-gated Na(+) channel from NaᵥAb crystal structure contains four fenestrations ²⁹. Classic antiarrhythmics, such as benzocaine and lidocaine, have low thermodynamic stability in the fenestrations; thus, they rapidly move to the inner vestibule and bind to their receptor sites ²⁵. The homologous tetramer, NaᵥAb, contains a Phenylalanine-203 at each fenestration, modulating its radial size ²⁹. A point mutation to an Alanine in Phenyalanine-203 results in a substantial increase in the binding affinity of flecainide to the channel at resting state; however, mutating the residue to Tryptophan resulted in a significant decrease in the binding affinity of flecainide ^{27,30}.

Crystal structures obtained during the NaᵥAb inactivated states are compatible with slow inactivation in eukaryotic Naᵥ channels, since NaᵥAb lack the fast inactivation particle ^{12,29}. The wild-type NaᵥAb, which was crystalized in its inactivated state, undergoes a few conformational changes upon inactivating; the voltage sensors are displaced, the selectivity filter narrows, and the activation gate collapses. During the inactivated state, reshaping of the fenestrations in NaᵥAb involves two opposing fenestrations growing larger and the other two becoming smaller ¹². This was compared to nearly identical fenestrations in NaᵥAb-I217C, which halts slow inactivation entry ¹².

Kaczmarski and Corry (2014) characterized the fenestrations of the mammalian skeletal muscle voltage-gated sodium channel, Naᵥ1.4, homology model built on NaᵥAb. Fenestrations found in Domains II-III and IV-I are narrower than the adjacent two (Domain I-II and III-IV) and their radial size is determined mainly by isoleucine and phenylalanine residues in S5s ²⁵. The cryo-EM structure solved for the American cockroach voltage-gated sodium channel (NaᵥPas) contains only one small fenestration formed by the pore-forming segments in Domains III-IV ³¹. The other three sides do not contain a lateral pore and are in isolation from the lipid bilayer. Homology models built on NaᵥPas for the neuronal voltage-gated sodium channel (Naᵥ1.2) and skeletal muscle voltage-gated sodium channel (Naᵥ1.4) show all four fenestrations constricting and dilating under dynamic simulations³¹.

The cryo-EM structure of rat Naᵥ1.5 (rNaᵥ1.5) was solved at 3.2-3.5 Å and was captured in a pre-activated state in which all four voltage sensors were partially activated; thus, the channel was partially inactivated ³². The four fenestrations were identified: Domain II-III fenestration was the largest compared to other fenestrations. Flecainide associates with residues in the central cavity via Domain II-III fenestrations. Other studies suggest that Domain III-IV fenestrations can also provide access for the drug ³³; however, this fenestration is relatively small compared to Domain II-III fenestration in rNaᵥ1.5.

Residues F1760 and Y1767 of human Naᵥ1.5 have been identified as binding sites for classic anti-arrhythmics and anti-convulsants ^{7,37,38}.

Many sodium channel-targeting compounds like Ranolazine, Phenytoin, Lidocaine, and other sodium channel blockers, which have almost identical effects at 100 μM ^{34,35}, bind with very low affinity to the fenestrations and preferentially stabilize slow inactivation by binding to their sites in the inner vestibule ²⁵.

Toxins like Batrachotoxin irreversibly bind to voltage-gated sodium channels (VGSC), immobilizing the channel in the open-state. This mechanism of action, however, may produce other adverse side effects that further exacerbate the pathophysiology of disease.

### SUMMARY OF THE INVENTION

In one aspect, the present invention relates to a method of inhibiting the inactivation of a Naᵥ1.5 voltage-gated sodium channel by contacting the Naᵥ1.5 voltage-gated sodium channel with a compound according to Formula I:

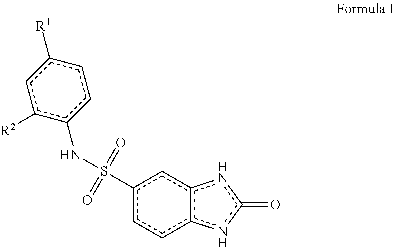

where R¹ may be halo, and

R² may be alkyl, alkenyl or alkynyl;

### or Formula II:

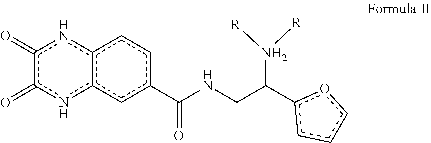

where R may each independently be alkyl, alkenyl or alkynyl.

In some embodiments, the inactivation may be slow inactivation, fast inactivation, or a combination thereof. In some embodiments, the inactivation may be slow inactivation and the compound may be a compound according to Formula I. In some embodiments, the inactivation m ay be fast inactivation and the compound may be a compound according to Formula II.

In some embodiments, the Naᵥ1.5 voltage-gated sodium channel may be in an inactivated state or a closed state. In some embodiments, the Naᵥ1.5 voltage-gated sodium channel may be in an inactivated state and the compound may be a compound according to Formula I. In some embodiments, the Naᵥ1.5 voltage-gated sodium channel may be in a closed state and the compound may be a compound according to Formula II.

In some embodiments, the compound may bind the Naᵥ1.5 voltage-gated sodium channel. In some embodiments, the compound may bind within a fenestration of the Naᵥ1.5 voltage-gated sodium channel.

In alternative aspects, the present invention relates to a method of treating a cardiovascular disease by administering a compound that inhibits the inactivation of a Naᵥ1.5 voltage-gated sodium channel to a subject in need thereof. In some embodiments, the cardiovascular disease may be Brugada Syndrome, cardiac arrhythmia disorders, progressive cardiac conduction disorder (PCCD), sick sinus syndrome, progressive familial heart block, atrial fibrillation, sudden infant death syndrome, dilated cardiomyopathy, myocardial ischemia/infarction or heart failure. In some embodiments, the subject may be a human. In some embodiments, the compound may be a compound according to Formula I:

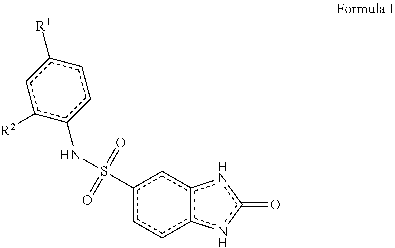

where R¹ is halo, and

R² is alkyl, alkenyl or alkynyl;

### or Formula II:

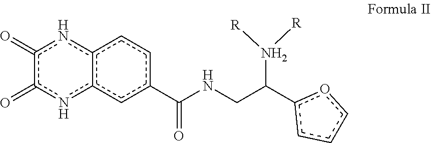

where R is each independently alkyl, alkenyl or alkynyl.

In alternative aspects, the present invention relates to a method of treating a cardiovascular disease by inhibiting the inactivation of a Naᵥ1.5 voltage-gated sodium channel in a subject in need thereof.

In alternative aspects, the present invention relates to a pharmaceutical composition comprising a compound according to Formula I:

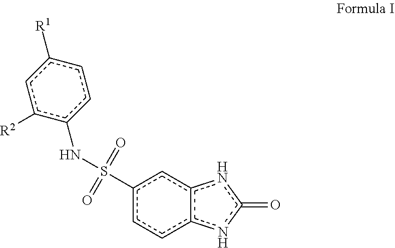

where R¹ is halo, and

R² is alkyl, alkenyl or alkynyl;

### or Formula II:

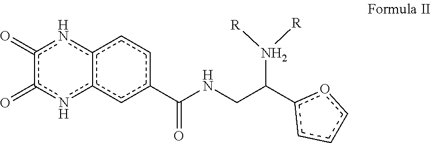

where R is each independently alkyl, alkenyl or alkynyl, in combination with a pharmaceutically acceptable carrier.

In alternative aspects, the present invention relates to a method of inhibiting the slow inactivation, the fast inactivation, or a combination thereof, of a Naᵥ1.5 voltage-gated sodium channel by contacting the Naᵥ1.5 voltage-gated sodium channel with a compound that binds within a fenestration of the Naᵥ1.5 voltage-gated sodium channel.

In an alternative aspect, the present invention decelerates the onset of fast inactivation of a Naᵥ1.5 voltage-gated sodium channel.

In some embodiments, the present invention relates to a method of treating a cardiovascular disease comprising administering a compound that binds within a fenestration of a Naᵥ1.5 voltage-gated sodium channel to a subject in need thereof.

In some embodiments, the compound may be selected from one or more of the compounds set forth in Table 2.

This summary of the invention does not necessarily describe all features of the invention.

### BRIEF DESCRIPTION OF THE DRAWINGS

These and other features of the invention will become more apparent from the following description in which reference is made to the appended drawings wherein:

FIGS. 1A-E show homology modelling of Naᵥ1.5. The pore-forming segments of Naᵥ1.5 homology model built on NaᵥPas is shown from the top view in FIG. 1A. FIGS. B-E show the external view of the fenestrations shown as dark pockets between the pore-forming helices. This eukaryotic homology model of the Na(+) channel partially accounts for the change in fenestration size, which changes in a state-dependent manner. The only fenestration in this model that remains dilated up until the central pore is Domain I-II (FIG. 1B) as opposed to the others (FIGS. 1C-E), which continue to narrow until they are fully obstructed at the central pore;

FIGS. 2A-B show the auto-docking results for Naᵥ1.5 homology model. The highest binding affinity mode is shown for three of the auto-docked compounds screened against NaᵥAb-Naᵥ1.5: ZINC40014265, ZINC38776626, ZINC38767647 where ZINC40014265 sits in Domain III-IV fenestration (FIG. 2A). Other compounds which bind to the fenestration (which have been characterized using electrophysiology as described below) are shown in FIG. 2B;

FIGS. 3A-C show the different sets of pulse protocols used to assess compound activity on voltage-gated sodium channels. Conductance was measured by a protocol (FIG. 3A) that depolarized the membrane from −100 mV to +80 mV in increments of +5 mV for 19 ms. Prior to the test pulse, channels were allowed to recover from fast inactivation at −130 mV for 197 ms. Slow inactivation (SI) was indirectly determined (FIG. 3B) by measuring use-dependence, which is a physiologically-relevant protocol. The protocol includes a series of 500 110 ms depolarizing pulses to 0 mV followed by a 55 ms-90 mV recovery pulse at a frequency 6 Hz. With repetitive depolarizations, Naᵥ1.5 channels accumulate into slow inactivation. FIG. 3C includes a double-pulse protocol used to directly measure slow inactivation following fast inactivation. This protocol was used to measured drug activity on Naᵥ1.1 and Naᵥ1.5 channels. Slow inactivation was measured after channels were pre-conditioned to the inactivation midpoint voltage of −65 mV or −90 mV for Naᵥ1.1 and Naᵥ1.5, respectively:

FIGS. 4A-G show the effects of the six compounds screened against Naᵥ1.5 using a single voltage-pulse protocol in transiently transfected HEK 293 cells. The six compounds have differential blocking affinities on peak I_{Na} at −20 mV as evident in the table showing normalized peak I_{Na} (normalized to I_{Na} at 0 μM) as a function of compound concentration (n=4-17). The only compound that does not reduce peak Na at concentrations above 10 μM is ZINC64470745;

FIGS. 5A-G shows the effects of the compounds on the current-voltage relationship screened against Naᵥ1.5 using a single voltage-pulse protocol in transiently transfected HEK 293 cells (n=4-17). A dose-response curve was fitted to the drug concentrations generating hill curves shown at the bottom of the figure. ZINC64470745 is the compound that blocks peak Na the least where the plateau of drug block on sodium current (−20%) is attained at concentrations above 10 μM. However, the highest IC₅₀s for peak I_{Na} block (potentiators) was found in ZINC40014265 and ZINC64470729 (Table 4);

FIGS. 6A-F show the effects of the compounds on voltage-dependence of conductance (n=4-16) screened against Naᵥ1.5 using the single voltage-pulse protocol in transiently transfected HEK 293 cells. All compounds had no effect on the conductance midpoint at any concentration:

FIGS. 7A-G show peak Na traces at −30 mV showing the effects of compounds at 50 μM screened against Naᵥ1.5 using the single voltage-pulse protocol in transiently transfected HEK 293 cells. The table is inclusive of all the fast inactivation onset T values in milliseconds at −30 mV, −10 mV, and +10 my (n=4-17). Most compounds decelerate the fast inactivation kinetics. Other compounds like ZINC38767171 decelerate fast inactivation kinetics, but not significantly. The compound that decelerates fast inactivation kinetics the most is ZINC64470737 at 50 μM;

FIGS. 8A-G show the effects of the compounds on use-dependence screened against Naᵥ1.5 using the single voltage-pulse protocol in transiently transfected HEK 293 cells. Normalized current is plotted as a function of time. All aromatic functional groups in the compounds tested resist the accumulation of use-dependence (which correlates with slow inactivation) compared to the aliphatic functional group present in ZINC39699427, which significantly increases Naᵥ1.5 use-dependence (n=4-16). There are no statistically significant differences in fast or slow use-dependence T values between the drug conditions. The dose-dependent curve shows the I_{Na} use-dependence percentage as a function of the drug concentration. Both ZINC40014265 and ZINC64470745 have the highest IC₅₀s (potentiators) compared to the other compounds as shown in Table 4;

FIG. 9 shows normalized inhibition of peak I_{Na} from a holding potential of −120 mV by nine compounds screened at 50 μM on a stable transfected HEK293 cells expressing either Naᵥ1.1 or Naᵥ1.5. Currents were measured using the double-pulse protocol described in FIG. 3C. All compounds have subtle effects on peak I_{Na} in Naᵥ1.1 and Naᵥ1.5 (n=4-11); however, ZINC12638098 blocks peak I_{Na} indifferently in both Naᵥ1.1 and Naᵥ1.5;

FIG. 10 shows normalized inhibition of peak I_{Na} from the mid-voltage potential held at 10 s (eliciting slow inactivation) at −65 mV and −90 mV for Naᵥ1.1 and Naᵥ1.5, respectively. The nine compounds screened were screened at 50 μM on a stable transfected HEK293 cells expressing either Naᵥ1.1 or Naᵥ1.5. Currents were measured using the double-pulse protocol described in FIG. 3C. All compounds have a larger block on peak I_{Na} when measured from mid-voltage potentials to a holding potential of −120 mV;

FIG. 11 shows normalized tau of inactivation from a holding potential of −120 mV for Naᵥ1.1 and Naᵥ1.5, respectively. The nine compounds screened were screened at 50 μM on a stable transfected HEK293 cells expressing either Naᵥ1.1 or Naᵥ1.5. Currents were measured using the double-pulse protocol described in FIG. 3C. Both ZINC12323863 and ZINC40014265 decelerate inactivation in Naᵥ1.5 compared to Naᵥ1.1. ZINC12323863 accelerates inactivation in Naᵥ1.1 compared to Naᵥ1.5;

FIG. 12 shows normalized tau of inactivation from the mid-voltage potential held at 10 s (eliciting slow inactivation) at −65 mV and −90 mV for Naᵥ1.1 and Naᵥ1.5, respectively, respectively. The nine compounds screened were screened at 50 μM on a stable transfected HEK293 cells expressing either Naᵥ1.1 or Naᵥ1.5. Currents were measured using the double-pulse protocol described in FIG. 3C. All compounds have no effect on tau of inactivation;

FIG. 13 shows normalized area under curve (AUC) of the activated sodium current measured from a holding potential of −120 mV. The ratio of control to compound AUC is plotted against the nine compounds screened at 50 μM on a stable transfected HEK293 cells expressing either Naᵥ1.1 or Naᵥ1.5. Currents were measured using the double-pulse protocol described in FIG. 3C. ZINC12323863 substantially reduces AUC ratio in Naᵥ1.1 compared to Naᵥ1.5. However, the AUC ratio is slightly increased by ZINC40014265 in Naᵥ1.5;

FIGS. 14A-E show the results of autodocking compounds against rNaᵥ1.5. FIGS. 14A-B show the residues that outline the outer diameter of Domain III-Domain IV (FIG. 14A) and Domain I-Domain IV (FIG. 14B) fenestrations, respectively. FIGS. 14C-D show the docking of nine compounds screened against rNaᵥ1.5 Domain III-Domain IV (FIG. 14C) and Domain IV-Domain I fenestrations (FIG. 14D), respectively. FIG. 14C shows docking of nine compounds against intact rNaV1.5 with all four domains included.

### DETAILED DESCRIPTION

The present disclosure provides, in part, methods and compounds for inhibiting inactivation of a Naᵥ1.5 voltage-gated sodium channel.

Voltage-gated sodium channels are transmembrane proteins that are responsible for electrical excitation in a variety of cells. Depolarization of the membrane results in opening or “activation” of a voltage-gated sodium channel, while repolarization results in closing or deactivation of the channel. Inactivation, the state in which the channel is unable or less able to conduct sodium current, may be achieved at depolarization, which occurs when the membrane potential rises above threshold potential (for example, above −50 mV). With maintained depolarization, inactivation leaves the channel temporarily refractory, i.e., incapable of passing current. Inactivation may also be achieved at rest (at relatively negative membrane potential, for example, about −50 mV). Inactivation of the channel may be fast or slow.

“Fast inactivation” generally lasts milliseconds and is mechanistically and pharmacologically distinct from slow inactivation. In some embodiments, fast inactivation of a voltage-gated sodium channel can range from about 50 milliseconds to about 500 milliseconds or any value in between, for example 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, or 500 milliseconds.

“Slow inactivation” of a voltage-gated sodium channel involves conformational changes in the selectivity filter, voltage sensors, and lateral pores known as “fenestrations” and can occur on the timescale of seconds to minutes. In some embodiments, slow inactivation of a voltage-gated sodium channel can last at least 160 seconds. In some embodiments, slow inactivation of a voltage-gated sodium channel can range from about 10 seconds to about 160 seconds or any value in between, for example, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, or 160 seconds. In some embodiments, slow inactivation of a voltage-gated sodium channel can range from about 10 seconds to about 60 seconds or any value in between, for example, 10, 15, 20, 25, 30, 35, 40, 45, 50, 55 or 60 seconds.

The fenestrations have been implicated as sites involved with slow inactivation development and with drug entry and binding. Conformational changes in the fenestration may also affect fast inactivation. Exacerbation of fast and slow inactivation is implied in various pathophysiological states associated with cardiac disease, such as myocardial ischemia/infarction or heart failure. Various SCN5a mutations can enhance both types of inactivation development in Naᵥ1.5.

Naᵥ1.5 is a cardiac voltage-gated sodium channel expressed predominantly in cardiac myocytes and in Purkinje fibers. The SCN5a gene, located in the short (p) arm of chromosome 3 at position 22.2, encodes the Naᵥ1.5 isoform. Human Naᵥ1.5 may have the following amino acid sequence:

(SEQ ID NO: 1)
10 20 30 40
MANFLLPRGT SSFRRFTRES LAAIEKRMAE KQARGSTTLQ
50 60 70 80
ESREGLPEEE APRPQLDLQA SKKLPDLYGN PPQELIGEPL
90 100 110 120
EDLDPFYSTQ KTFIVLNKGK TIFRFSATNA LYVLSPFHPI
130 140 150 160
RRAAVKILVH SLFNMLIMCT ILTNCVFMAQ HDPPPWTKYV
170 180 190 200
EYTFTAIYTF ESLVKILARG FCLHAFTFLR DPWNWLDFSV
210 220 230 240
IIMAYTTEFV DLGNVSALRT FRVLRALKTI SVISGLKTIV
250 260 270 280
GALIQSVKKL ADVMVLTVFC LSVFALIGLQ LFMGNLRHKC
290 300 310 320
VRNFTALNGT NGSVEADGLV WESLDLYLSD PENYLLKNGT
330 340 350 360
SDVLLCGNSS DAGTCPEGYR CLKAGENPDH GYTSFDSFAW
370 380 390 400
AFLALFRLMT QDCWERLYQQ TLRSAGKIYM IFFMLVIFLG
410 420 430 440
SFYLVNLILA VVAMAYEEQN QATIAETEEK EKRFQEAMEM
450 460 470 480
LKKEHEALTI RGVDTVSRSS LEMSPLAPVN SHERRSKRRK
490 500 510 520
RMSSGTEECG EDRLPKSDSE DGPRAMNHLS LTRGLSRTSM
530 540 550 560
KPRSSRGSIF TFRRRDLGSE ADFADDENST AGESESHHTS
570 580 590 600
LLVPWPLRRT SAQGQPSPGT SAPGHALHGK KNSTVDCNGV
610 620 630 640
VSLLGAGDPE ATSPGSHLLR PVMLEHPPDT TTPSEEPGGP
650 660 670 680
QMLTSQAPCV DGFEEPGARQ RALSAVSVLT SALEELEESR
690 700 710 720
HKCPPCWNRL AQRYLIWECC PLWMSIKQGV KLVVMDPFTD
730 740 750 760
LTITMCIVLN TLFMALEHYN MTSEFEEMLQ VGNLVFTGIF
770 780 790 800
TAEMTFKIIA LDPYYYFQQG WNIFDSIIVI LSLMELGLSR
810 820 830 840
MSNLSVLRSF RLLRVFKLAK SWPTLNTLIK IIGNSVGALG
850 860 870 880
NLTLVLAIIV FIFAVVGMQL FGKNYSELRD SDSGLLPRWH
890 900 910 920
MMDFFHAFLI IFRILCGEWI ETMWDCMEVS GQSLCLLVFL
930 940 950 960
LVMVIGNLVV LNLFLALLLS SFSADNLTAP DEDREMNNLQ
970 980 990 1000
LALARIQRGL RFVKRTTWDF CCGLLRQRPQ KPAALAAQGQ
1010 1020 1030 1040
LPSCIATPYS PPPPETEKVP PTRKETRFEE GEQPGQGTPG
1050 1060 1070 1080
DPEPVCVPIA VAESDTDDQE EDEENSLGTE EESSKQQESQ
1090 1100 1110 1120
PVSGGPEAPP DSRTWSQVSA TASSEAEASA SQADWRQQWK
1130 1140 1150 1160
AEPQAPGCGE TPEDSCSEGS TADMTNTAEL LEQIPDLGQD
1170 1180 1190 1200
VKDPEDCFTE GCVRRCPCCA VDTTQAPGKV WWRLRKTCYH
1210 1220 1230 1240
IVEHSWFETF IIFMILLSSG ALAFEDIYLE ERKTIKVLLE
1250 1260 1270 1280
YADKMFTYVF VLEMLLKWVA YGFKKYFTNA WCWLDFLIVD
1290 1300 1310 1320
VSLVSLVANT LGFAEMGPIK SLRTLRALRP LRALSRFEGM
1330 1340 1350 1360
RVVVNALVGA IPSIMNVLLV CLIFWLIFSI MGVNLFAGKF
1370 1380 1390 1400
GRCINQTEGD LPLNYTIVNN KSQCESLNLT GELYWTKVKV
1410 1420 1430 1440
NFDNVGAGYL ALLQVATFKG WMDIMYAAVD SRGYEEQPQW
1450 1460 1470 1480
EYNLYMYIYF VIFIIFGSFF TLNLFIGVII DNFNQQKKKL
1490 1500 1510 1520
GGQDIFMTEE QKKYYNAMKK LGSKKPQKPI PRPLNKYQGF
1530 1540 1550 1560
IFDIVTKQAF DVTIMFLICL NMVTMMVETD DQSPEKINIL
1570 1580 1590 1600
AKINLLFVAI FTGECIVKLA ALRHYYFTNS WNIFDFVVVI
1610 1620 1630 1640
LSIVGTVLSD IIQKYFFSPT LFRVIRLARI GRILRLIRGA
1650 1660 1670 1680
KGIRTLLFAL MMSLPALFNI GLLLFLVMFI YSIFGMANFA
1690 1700 1710 1720
YVKWEAGIDD MFNFQTFANS MLCLFQITTS AGWDGLLSPI
1730 1740 1750 1760
LNTGPPYCDP TLPNSNGSRG DCGSPAVGIL FFTTYIIISF
1770 1780 1790 1800
LIVVNMYIAI ILENFSVATE ESTEPLSEDD FDMFYEIWEK
1810 1820 1830 1840
FDPEATQFIE YSVLSDFADA LSEPLRIAKP NQISLINMDL
1850 1860 1870 1880
PMVSGDRIHC MDILFAFTKR VLGESGEMDA LKIQMEEKFM
1890 1900 1910 1920
AANPSKISYE PITTTLRRKH EEVSAMVIQR AFRRHLLQRS
1930 1940 1950 1960
LKHASFLFRQ QAGSGLSEED APEREGLIAY VMSENFSRPL
1970 1980 1990 2000
GPPSSSSISS TSFPPSYDSV TRATSDNLQV RGSDYSHSED
2010
LADFPPSPDR DRESIV

In some embodiments, a Naᵥ1.5 voltage-gated sodium channel may include without limitation Naᵥ1.5 from mouse, rat, dog, sheep, cow, etc. In some embodiments, a Naᵥ1.5 voltage-gated sodium channel may include an amino acid sequence having at least 90% sequence identity to SEQ ID NO: 1, for example, 90% to 100% sequence identity or any value in between such as 90%, 91%, 92%, 93%, 94%, 95%, 96%, 97%, 98% or 99% sequence identity to SEQ ID NO: 1.

In some embodiments, the present disclosure provides a compound that can inhibit the inactivation of a Naᵥ1.5 voltage-gated sodium channel. As used herein, inhibiting the inactivation may include slowing inactivation and/or decelerating entry into fast inactivation of a Naᵥ1.5 voltage-gated sodium channel.

In some embodiments, the compound in accordance with the present disclosure that can inhibit the inactivation of a Naᵥ1.5 voltage-gated sodium channel may have the chemical structure set forth in Formula I:

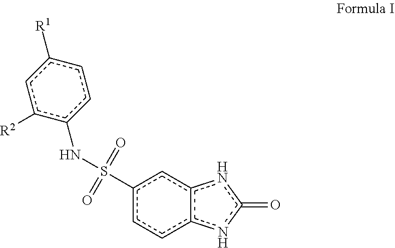

where R¹ may be halo; and

R² may be alkyl, alkenyl or alkynyl.

In some embodiments, the compound in accordance with the present disclosure that can inhibit the inactivation of a Naᵥ1.5 voltage-gated sodium channel may have the chemical structure set forth in Formula II:

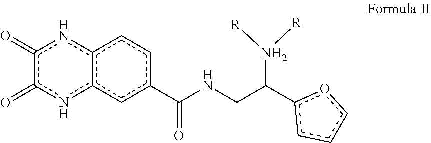

where R may each independently be alkyl, alkenyl or alkynyl.

As used herein the singular forms “a”, “and”, and “the” include plural referents unless the context clearly dictates otherwise. For example, “a compound” refers to one or more of such compounds, while “the Naᵥ1.5 voltage-gated sodium channel” includes a particular polypeptide as well as other family member equivalents thereof as known to those skilled in the art.

Throughout this application, it is contemplated that the term “compound” or “compounds” refers to the compounds discussed herein and includes precursors and derivatives of the compounds, including acyl-protected derivatives, and pharmaceutically acceptable salts of the compounds, precursors, and derivatives. The invention also includes prodrugs of the compounds, pharmaceutical compositions including the compounds and a pharmaceutically acceptable carrier, and pharmaceutical compositions including prodrugs of the compounds and a pharmaceutically acceptable carrier.

The compounds of the present disclosure may contain one or more asymmetric centers and can thus occur as racemates and racemic mixtures, single enantiomers, diastereomeric mixtures and individual diastereomers. Additional asymmetric centers may be present depending upon the nature of the various substituents on the molecule. Each such asymmetric center will independently produce two optical isomers and it is intended that all of the possible optical isomers and diastereomers in mixtures and as pure or partially purified compounds are included within the ambit of this invention. Any formulas, structures or names of compounds described in this specification that do not specify a particular stereochemistry are meant to encompass any and all existing isomers as described above and mixtures thereof in any proportion. When stereochemistry is specified, the invention is meant to encompass that particular isomer in pure form or as part of a mixture with other isomers in any proportion.

“Alkyl” refers to a straight or branched hydrocarbon chain group consisting solely of carbon and hydrogen atoms, containing no unsaturation and including, for example, from one to ten carbon atoms, such as 1, 2, 3, 4, 5, 6, 7, 8, 9, or 10 carbon atoms, and which is attached to the rest of the molecule by a single bond. In alternative embodiments, the alkyl group may contain from one to eight carbon atoms, such as 1, 2, 3, 4, 5, 6, 7, or 8 carbon atoms. In alternative embodiments, the alkyl group may contain from one to six carbon atoms, such as 1, 2, 3, 4, 5, or 6 carbon atoms. Unless stated otherwise specifically in the specification, the alkyl group may be optionally substituted by one or more substituents as described herein. Unless stated otherwise specifically herein, it is understood that the substitution can occur on any carbon of the alkyl group. In some embodiments, the alkyl may be methyl, ethyl, propyl, isopropyl, butyl, tert-butyl, pentyl, or isopentyl.

“Alkenyl” refers to a straight or branched hydrocarbon chain group consisting solely of carbon and hydrogen atoms, containing at least one double bond and including, for example, from two to ten carbon atoms, such as 2, 3, 4, 5, 6, 7, 8, 9, or 10 carbon atoms, and which is attached to the rest of the molecule by a single bond or a double bond. In alternative embodiments, the alkenyl group may contain from two to eight carbon atoms, such as 2, 3, 4, 5, 6, 7, or 8 carbon atoms. In alternative embodiments, the alkenyl group may contain from three to six carbon atoms, such as 3, 4, 5, or 6 carbon atoms. Unless stated otherwise specifically in the specification, the alkenyl group may be optionally substituted by one or more substituents as described herein. Unless stated otherwise specifically herein, it is understood that the substitution can occur on any carbon of the alkenyl group.

“Alkynyl” refers to a straight or branched hydrocarbon chain group consisting solely of carbon and hydrogen atoms, containing at least one triple bond and including, for example, from two to ten carbon atoms, such as 2, 3, 4, 5, 6, 7, 8, 9, or 10 carbon atoms. In alternative embodiments, the alkynyl group may contain from two to eight carbon atoms, such as 2, 3, 4, 5, 6, 7, or 8 carbon atoms. In alternative embodiments, the alkynyl group may contain from three to six carbon atoms, such as 3, 4, 5, or 6 carbon atoms. Unless stated otherwise specifically in the specification, the alkynyl group may be optionally substituted by one or more substituents as described herein.

“Halo” refers to bromo, chloro, fluoro, iodo, etc. In some embodiments, suitable halogens include bromine, iodine, fluorine or chlorine.

“Optional” or “optionally” means that the subsequently described event of circumstances may or may not occur, and that the description includes instances where said event or circumstance occurs one or more times and instances in which it does not. For example, “optionally substituted alkyl” means that the alkyl group may or may not be substituted and that the description includes both substituted alkyl groups and alkyl groups having no substitution, and that said alkyl groups may be substituted one or more times. Examples of suitable optional substituents include, without limitation, O, N, S, etc.

In some embodiments, the compound in accordance with the present disclosure that can inhibit the inactivation of a Naᵥ1.5 voltage-gated sodium channel may be selected from one or more of the compounds set forth in Table 2.

In some embodiments, a compound in accordance with the present disclosure may specifically bind a Naᵥ1.5 voltage-gated sodium channel. In some embodiments, a compound in accordance with the present disclosure that specifically binds a Naᵥ1.5 voltage-gated sodium channel may specifically bind to the “inactivated” state i.e. maintained in the “inactivated” configuration for a period of time depending on whether the inactivation is fast or slow, of the Naᵥ1.5 voltage-gated sodium channel. Specific binding to the inactivated state of the Naᵥ1.5 voltage-gated sodium channel may be determined by any suitable methods, such as the single-pulse use dependence methods described herein. In some embodiments, a compound in accordance with the present disclosure that specifically binds a Naᵥ1.5 voltage-gated sodium channel in the inactivated state may inhibit the development of slow inactivation. In some embodiments, a compound in accordance with the present disclosure that specifically binds a Naᵥ1.5 voltage-gated sodium channel in the inactivated state may be a compound according to Formula I.

In some embodiments, a compound in accordance with the present disclosure that specifically binds a Naᵥ1.5 voltage-gated sodium channel may specifically bind to the “closed” or “deactivated” state of the Naᵥ1.5 voltage-gated sodium channel. Specific binding to the closed state of the Naᵥ1.5 voltage-gated sodium channel may be determined by any suitable methods, such as the high-throughput double pulse methods described herein. In some embodiments, a compound in accordance with the present disclosure that specifically binds a Naᵥ1.5 voltage-gated sodium channel in the closed state may decelerate fast inactivation kinetics (i.e., inhibit the development of fast inactivation) in comparison, for example, to a neuronal voltage-gated sodium channel (such as Naᵥ1.1). In some embodiments, a compound in accordance with the present disclosure that specifically binds a Naᵥ1.5 voltage-gated sodium channel in the closed state may be a compound according to Formula II.

In alternative embodiments, a compound in accordance with the present disclosure may specifically bind within a fenestration of the Naᵥ1.5 voltage-gated sodium channel. In some embodiments, a compound in accordance with the present disclosure that specifically binds within a fenestration of the Naᵥ1.5 voltage-gated sodium channel may bind within the fenestration of any one of Domains I-II, II-III, II-IV, or IV-I. In some embodiments, a compound in accordance with the present disclosure that specifically binds within a fenestration of the Naᵥ1.5 voltage-gated sodium channel may bind within the fenestration of III-IV. In some embodiments, a compound in accordance with the present disclosure that specifically binds within a fenestration of the Naᵥ1.5 voltage-gated sodium channel may bind within the fenestration of IV-I. In some embodiments, a compound in accordance with the present disclosure that binds within a fenestration of the Naᵥ1.5 voltage-gated sodium channel may bind to one or more of the residues set out in Table 1. In some embodiments, a compound in accordance with the present disclosure that binds within a fenestration of the Naᵥ1.5 voltage-gated sodium channel may bind to one or more of the residues, other than F1760 and Y1767, set out in Table 1.

In some embodiments, a compound that binds a Naᵥ1.5 voltage-gated sodium channel may be selected from one or more of the compounds set out in Table 2.

In some embodiments, a compound in accordance with the present disclosure that binds a Naᵥ1.5 voltage-gated sodium channel may inhibit the ability of the Naᵥ1.5 voltage-gated sodium channel to undergo structural rearrangements leading to slow inactivation. In some embodiments, a compound in accordance with the present disclosure that binds a Naᵥ1.5 voltage-gated sodium channel may inhibit the ability of the Naᵥ1.5 voltage-gated sodium channel to undergo structural rearrangements leading to deceleration of fast inactivation onset. In some embodiments, a compound in accordance with the present disclosure that binds a Naᵥ1.5 voltage-gated sodium channel may inhibit the ability of the Naᵥ1.5 voltage-gated sodium channel to undergo fast and/or slow inactivation onset and/or stabilization. In some embodiments, a compound in accordance with the present disclosure that binds a Naᵥ1.5 voltage-gated sodium channel may inhibit the ability of the Naᵥ1.5 voltage-gated sodium channel fenestrations to constrict and/or dilate during slow inactivation. In some embodiments, a compound in accordance with the present disclosure that binds a Naᵥ1.5 voltage-gated sodium channel may inhibit loss-of-function in a WT-Naᵥ1.5 channel. In some embodiments, a compound in accordance with the present disclosure may be any one of ZINC40014265 or ZINC12323863 and may decelerate fast inactivation kinetics at depolarized voltages. In some embodiments, a compound in accordance with the present disclosure may be ZINC64470745 and may inhibit slow inactivation. Without being bound to any particular theory, the aromatic functional groups in ZINC64470745 may resist slow inactivation at a stimulation frequency of 6 Hz compared to ZINC39699427 which contains an aliphatic functional group. Without being bound to any particular theory, the branched methyl chains in ZINC40014265 may resist fast inactivation, making this compound highly selective in its ability to target fast inactivation in Naᵥ1.5 compared to Naᵥ1.1.

In some embodiments, a compound that inhibits inactivation of the Naᵥ1.5 voltage-gated sodium channel may inhibit the ability of the Naᵥ1.5 voltage-gated sodium channel to undergo structural rearrangements leading to slow inactivation. In some embodiments, a compound that inhibits inactivation of the Naᵥ1.5 voltage-gated sodium channel may inhibit the ability of the Naᵥ1.5 voltage-gated sodium channel to undergo structural rearrangements leading to deceleration of fast inactivation onset. In some embodiments, a compound that inhibits inactivation of the Naᵥ1.5 voltage-gated sodium channel may inhibit the ability of the Naᵥ1.5 voltage-gated sodium channel to undergo fast and/or slow inactivation onset and/or stabilization. In some embodiments, a compound that inhibits inactivation of the Naᵥ1.5 voltage-gated sodium channel may inhibit the ability of the Naᵥ1.5 voltage-gated sodium channel fenestrations to constrict and/or dilate during slow inactivation. In some embodiments, a compound that inhibits inactivation of the Naᵥ1.5 voltage-gated sodium channel may inhibit loss-of-function in a WT-Naᵥ1.5 channel.

By “specifically binds” is meant a compound that binds a Naᵥ1.5 voltage-gated sodium channel but does not substantially bind other molecules in a sample. In some embodiments, by “specifically binds” is meant a compound that binds a fenestration of a Naᵥ1.5 voltage-gated sodium channel but does not substantially bind elsewhere on the Naᵥ1.5 voltage-gated sodium channel. In some embodiments, by “specifically binds” is meant a compound that binds the “inactivated” state of a Naᵥ1.5 voltage-gated sodium channel but does not substantially bind the Naᵥ1.5 voltage-gated sodium channel in the “closed” state. In some embodiments, by “specifically binds” is meant a compound that binds the “closed” state of a Naᵥ1.5 voltage-gated sodium channel but does not substantially bind the Naᵥ1.5 voltage-gated sodium channel in the “inactivated” state.

By “inhibit” “inhibition” or “inhibiting” is meant to prevent, control, decrease, reduce, reverse, slow or otherwise interfere with slow inactivation, or decelerate fast inactivation, of a voltage-gated sodium channel by at least about 10% to at least about 100%, or any value therebetween for example about 10%, 15%, 20%, 25%, 30%, 35%, 40%, 45%, 50%, 55%, 60%, 65%, 70%, 75%, 80%, 85%, 90%, 95%, 99%, or 100% in the presence of a test compound, when compared to a control compound that is known to have no effect on slow inactivation and/or fast inactivation, as appropriate, or in the absence of the test compound. In alternative embodiments, by “inhibit” “inhibition” or “inhibiting” is meant to prevent, control, decrease, reduce, reverse, slow or otherwise interfere with the slow inactivation, or decelerate fast inactivation, of a voltage-gated sodium channel by at least about 1.0 fold to about 10-fold, or any value therebetween for example about 1.0, 1.5, 2.0, 2.5, 3.0, 3.5, 4.0, 4.5, 5.0, 5.5, 6.0, 6.5, 7.0, 7.5, 8.0, 8.5, 9, 0, 9.5, or 10-fold, in the presence of a test compound, when compared to a control compound that is known to have no effect on slow inactivation, or in the absence of the test compound.

By “decelerate” “deceleration” or “decelerating” is meant to prevent, control, decrease, reduce, reverse, slow or otherwise interfere with fast inactivation of a voltage-gated sodium channel by at least about 10% to at least about 100%, or any value therebetween for example about 10%, 15%, 20%, 25%, 30%, 35%, 40%, 45%, 50%, 55%, 60%, 65%, 70%, 75%, 80%, 85%, 90%, 95%, 99%, or 100% in the presence of a test compound, when compared to a control compound that is known to have no effect on slow inactivation, or in the absence of the test compound. In alternative embodiments, by “decelerate” “deceleration” or “decelerating” is meant to prevent, control, decrease, reduce, reverse, slow or otherwise interfere with the fast inactivation of a voltage-gated sodium channel by at least about 2-fold to about 10-fold, or any value therebetween for example about 2.0, 2.5, 3.0, 3.5, 4.0, 4.5, 5.0, 5.5, 6.0, 6.5, 7.0, 7.5, 8.0, 8.5, 9.0, 9.5, or 10-fold, in the presence of a test compound, when compared to a control compound that is known to have no effect on fast inactivation, or in the absence of the test compound.

The effect of a compound on fast and/or slow inactivation and/or quantification thereof, may be determined using any suitable technique, for example by analyzing channel use-dependence. For example, by increasing stimulation frequency, sodium channel availability is reduced due to slow inactivation. Accordingly, a compound capable of preventing slow inactivation, for example, may resist channel use-dependence as a function of frequency.

In some embodiments, the present disclosure provides a method of inhibiting a Naᵥ1.5 voltage-gated sodium channel by inactivating the Naᵥ1.5 voltage-gated sodium channel.

In some embodiments, the present disclosure provides a method of inactivating a Naᵥ1.5 voltage-gated sodium channel by contacting the Naᵥ1.5 voltage-gated sodium channel with a compound as described herein.

In some embodiments, a compound that binds a Naᵥ1.5 voltage-gated sodium channel may be useful to treat a cardiovascular disease. In some embodiments, a compound as set forth in Table 2 may be useful to treat a cardiovascular disease in a subject in need thereof.

By “cardiovascular disease” or “cardiac disease” is meant a condition that may, if untreated, lead to cardiac arrest, sudden infant death syndrome (SIDS), and/or sudden cardiac death (SCD). In some embodiments, a “cardiovascular disease” includes inherited or non-inherited conditions that generate irregular heart rhythms, known as “arrhythmias”. These arrhythmias may be exacerbated by myocardial ischemia, myocardial infarction and/or heart failure, which may enhance fast and slow inactivation in Naᵥ1.5 voltage-gated sodium channels. These biophysical shifts causing loss-of-function in Naᵥ1.5 accompany myocardial ischemia/infarction and/or genetic diseases like progressive cardiac conduction disorder and Brugada syndrome etc. In some embodiments, a “cardiovascular disease” includes a disorder resulting from loss-of-function in Naᵥ1.5. Accordingly, a cardiovascular disease, as used herein, includes without limitation Brugada Syndrome, cardiac arrhythmia disorders, progressive cardiac conduction disorder (PCCD), sick sinus syndrome, progressive familial heart block, atrial fibrillation, sudden infant death syndrome, dilated cardiomyopathy, myocardial ischemia/infarction or heart failure.

As used herein, a “subject” may be a human, non-human primate, rat, mouse, cow, horse, pig, sheep, goat, dog, cat, etc. The subject may be a clinical patient, a clinical trial volunteer, an experimental animal, etc. The subject may be suspected of having or at risk for having a cardiovascular disease or be diagnosed with cardiovascular disease. In some cases, the subject may have relapsed after treatment for cardiovascular disease.

Pharmaceutical compositions including compounds according to the present disclosure, or for use according to the present disclosure, are contemplated as being within the scope of the invention. In some embodiments, pharmaceutical compositions including an effective amount of a compound of Formula (I) or Formula (II) or as set forth in Table 2, are provided.

The compounds of Formula (I) or Formula (II) or as set forth in Table 2, and their pharmaceutically acceptable salts, enantiomers, solvates, or derivatives may be useful because they may have pharmacological activity in animals, including humans. In some embodiments, one or more of the compounds according to the present disclosure may be stable in plasma, when administered to a subject, such as a human.

In general, a compound according to the present disclosure may be administered to a subject in need thereof, or by contacting a cell or a sample, for example, with a pharmaceutical composition comprising a therapeutically effective amount of the compound according to Formula (I) or Formula (II) or as set forth in Table 2.

In some embodiments, a compound according to the present disclosure, or for use according to the present disclosure, may be provided in combination with any other active agents or pharmaceutical compositions where such combined therapy may be useful to inhibit the inactivation of a Naᵥ1.5 voltage-gated sodium channel, for example, to treat a cardiovascular disease or any condition described herein. In some embodiments, a compound according to the present disclosure, or for use according to the present disclosure, may be provided in combination with one or more agents useful in the prevention or treatment of a cardiovascular disease.

It is to be understood that combination of compounds according to the present disclosure, or for use according to the present disclosure, with agents useful for the treatment of a cardiovascular disease is not limited to the examples described herein, but may include combination with any agent useful for the treatment of a cardiovascular disease. Combination of compounds according to the present disclosure, or for use according to the present disclosure, and other agents useful for the treatment of a cardiovascular disease may be administered separately or in conjunction. The administration of one agent may be prior to, concurrent to, or subsequent to the administration of other agent(s).

In alternative embodiments, a compound according to the present disclosure may be supplied as a “prodrug” or as protected forms, which release the compound after administration to a subject. For example, a compound may carry a protective group which is split off by hydrolysis in body fluids, e.g., in the bloodstream, thus releasing the active compound or is oxidized or reduced in body fluids to release the compound. Accordingly, a “prodrug” is meant to indicate a compound that may be converted under physiological conditions or by solvolysis to a biologically active compound of the present disclosure. Thus, the term “prodrug” refers to a metabolic precursor of a compound of the present disclosure that is pharmaceutically acceptable. A prodrug may be inactive when administered to a subject in need thereof, but may be converted in vivo to an active compound of the present disclosure. Prodrugs are typically rapidly transformed in vivo to yield the parent compound of the present disclosure, for example, by hydrolysis in blood. The prodrug compound often offers advantages of solubility, tissue compatibility or delayed release in a subject.

The term “prodrug” is also meant to include any covalently bonded carriers which release the active compound of the present disclosure in vivo when such prodrug is administered to a subject. Prodrugs of a compound of the present disclosure may be prepared by modifying functional groups present in the compound of the present disclosure in such a way that the modifications are cleaved, either in routine manipulation or in vivo, to the parent compound of the present disclosure. Prodrugs include compounds of the present disclosure where a hydroxy, amino or mercapto group is bonded to any group that, when the prodrug of the compound of the present disclosure is administered to a mammalian subject, cleaves to form a free hydroxy, free amino or free mercapto group, respectively. Examples of prodrugs include, but are not limited to, acetate, formate and benzoate derivatives of alcohol and acetamide, formamide, and benzamide derivatives of amine functional groups in one or more of the compounds of the present disclosure and the like.

A discussion of prodrugs may be found in “Smith and Williams' Introduction to the Principles of Drug Design,” H. J. Smith, Wright, Second Edition, London (1988); Bundgard, H., Design of Prodrugs (1985), pp. 7-9, 21-24 (Elsevier, Amsterdam); The Practice of Medicinal Chemistry, Camille G. Wermuth et al., Ch 31, (Academic Press, 1996); A Textbook of Drug Design and Development, P. Krogsgaard-Larson and H. Bundgaard, eds. Ch 5, pgs 113 191 (Harwood Academic Publishers, 1991); Higuchi, T., et al., “Pro-drugs as Novel Delivery Systems,” A.C.S. Symposium Series, Vol. 14; or in Bioreversible Carriers in Drug Design, ed. Edward B. Roche, American Pharmaceutical Association and Pergamon Press, 1987.

Compounds according to the present disclosure, or for use according to the present disclosure, may be provided alone or in combination with other compounds in the presence of a liposome, a nanoparticle, an adjuvant, or any pharmaceutically acceptable carrier, diluent or excipient, in a form suitable for administration to a subject such as a mammal, for example, humans, cattle, sheep, etc. If desired, treatment with a compound according to the present disclosure may be combined with more traditional and existing therapies for the therapeutic indications described herein. Compounds according to the present disclosure may be provided chronically or intermittently. “Chronic” administration refers to administration of the compound(s) in a continuous mode as opposed to an acute mode, so as to maintain the initial therapeutic effect (activity) for an extended period of time. “Intermittent” administration is treatment that is not consecutively done without interruption, but rather is cyclic in nature. The terms “administration,” “administrable,” or “administering” as used herein should be understood to mean providing a compound of the present disclosure to the subject in need of treatment.

“Pharmaceutically acceptable carrier, diluent or excipient” may include, without limitation, any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier that has been approved, for example, by the United States Food and Drug Administration or other governmental agency as being acceptable for use in humans or domestic animals.

A compound of the present disclosure may be administered in the form of a pharmaceutically acceptable salt. In such cases, pharmaceutical compositions in accordance with this present disclosure may comprise a salt of such a compound, preferably a physiologically acceptable salt, which are known in the art. In some embodiments, the term “pharmaceutically acceptable salt” as used herein means an active ingredient comprising compounds of Formula (I) or Formula (II) or as set forth in Table 2, used in the form of a salt thereof, particularly where the salt form confers on the active ingredient improved pharmacokinetic properties as compared to the free form of the active ingredient or other previously disclosed salt form.

A “pharmaceutically acceptable salt” may include both acid and base addition salts. A “pharmaceutically acceptable acid addition salt” refers to those salts which retain the biological effectiveness and properties of the free bases, which are not biologically or otherwise undesirable, and which may be formed with inorganic acids such as hydrochloric acid, hydrobromic acid, sulfuric acid, nitric acid, phosphoric acid and the like, and organic acids such as acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like.

A “pharmaceutically acceptable base addition salt” refers to those salts which may retain the biological effectiveness and properties of the free acids, which may not be biologically or otherwise undesirable. These salts may be prepared from addition of an inorganic base or an organic base to the free acid. Salts derived from inorganic bases may include, but are not limited to, the sodium, potassium, lithium, ammonium, calcium, magnesium, iron, zinc, copper, manganese, aluminum salts and the like. Preferred inorganic salts may be the ammonium, sodium, potassium, calcium, and magnesium salts. Salts derived from organic bases may include, but are not limited to, salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, 2-dimethylaminoethanol, 2-diethylaminoethanol, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, polyamine resins and the like. Particularly preferred organic bases may be isopropylamine, diethylamine, ethanolamine, trimethylamine, dicyclohexylamine, choline and caffeine.

Thus, the term “pharmaceutically acceptable salt” encompasses all acceptable salts including but not limited to acetate, lactobionate, benzenesulfonate, laurate, benzoate, malate, bicarbonate, maleate, bisulfate, mandelate, bitartarate, mesylate, borate, methylbromide, bromide, methylnitrite, calcium edetate, methylsulfate, camsylate, mucate, carbonate, napsylate, chloride, nitrate, clavulanate, N-methylglucamine, citrate, ammonium salt, dihydrochloride, oleate, edetate, oxalate, edisylate, pamoate (embonate), estolate, palmitate, esylate, pantothenate, fumarate, phosphate/diphosphate, gluceptate, polygalacturonate, gluconate, salicylate, glutame, stearate, glycollylarsanilate, sulfate, hexylresorcinate, subacetate, hydradamine, succinate, hydrobromide, tannate, hydrochloride, tartrate, hydroxynaphthoate, teoclate, iodide, tosylate, isothionate, triethiodide, lactate, panoate, valerate, and the like.

Pharmaceutically acceptable salts of a compound of the present disclosure may be used as a dosage for modifying solubility or hydrolysis characteristics, or may be used in sustained release or prodrug formulations. Also, pharmaceutically acceptable salts of a compound of this present disclosure may include those formed from cations such as sodium, potassium, aluminum, calcium, lithium, magnesium, zinc, and from bases such as ammonia, ethylenediamine, N-methyl-glutamine, lysine, arginine, ornithine, choline, N,N′-dibenzylethylene-diamine, chloroprocaine, diethanolamine, procaine, N-benzylphenethyl-amine, diethylamine, piperazine, tris(hydroxymethyl)aminomethane, and tetramethylammonium hydroxide.

Pharmaceutical formulations may typically include one or more carriers acceptable for the mode of administration of the preparation, be it by injection, inhalation, topical administration, lavage, or other modes suitable for the selected treatment. Suitable carriers may be those known in the art for use in such modes of administration.

Suitable pharmaceutical compositions may be formulated by means known in the art and their mode of administration and dose determined by the skilled practitioner. For parenteral administration, a compound may be dissolved in sterile water or saline or a pharmaceutically acceptable vehicle used for administration of non-water-soluble compounds such as those used for vitamin K. For enteral administration, the compound may be administered in a tablet, capsule or dissolved in liquid form. The table or capsule may be enteric coated, or in a formulation for sustained release. Many suitable formulations are known, including, polymeric or protein microparticles encapsulating a compound to be released, ointments, gels, hydrogels, or solutions which can be used topically or locally to administer a compound. A sustained release patch or implant may be employed to provide release over a prolonged period of time. Many techniques known to skilled practitioners are described in Remington: The Science & Practice of Pharmacy by Alfonso Gennaro, 20^{th} ed., Williams & Wilkins, (2000). Formulations for parenteral administration may, for example, contain excipients, polyalkylene glycols such as polyethylene glycol, oils of vegetable origin, or hydrogenated naphthalenes. Biocompatible, biodegradable lactide polymer, lactide/glycolide copolymer, or polyoxyethylene-polyoxypropylene copolymers may be used to control the release of a compound. Other potentially useful parenteral delivery systems for modulatory compounds may include ethylene-vinyl acetate copolymer particles, osmotic pumps, implantable infusion systems, and liposomes. Formulations for inhalation may contain excipients, for example, lactose, or may be aqueous solutions containing, for example, polyoxyethylene-9-lauryl ether, glycocholate and deoxycholate, or may be oily solutions for administration in the form of nasal drops, or as a gel.

A compound or a pharmaceutical composition according to the present disclosure may be administered by oral or non-oral, e.g., intramuscular, intraperitoneal, intravenous, intracisternal injection or infusion, subcutaneous injection, transdermal or transmucosal routes. In some embodiments, a compound or pharmaceutical composition in accordance with this present disclosure or for use in this present disclosure may be administered by means of a medical device or appliance such as an implant, graft, prosthesis, stent, etc. Implants may be devised which are intended to contain and release such compounds or compositions. An example would be an implant made of a polymeric material adapted to release the compound over a period of time. A compound may be administered alone or as a mixture with a pharmaceutically acceptable carrier e.g., as solid formulations such as tablets, capsules, granules, powders, etc.; liquid formulations such as syrups, injections, etc.; injections, drops, suppositories. In some embodiments, compounds or pharmaceutical compositions in accordance with this present disclosure or for use in this present disclosure may be administered by inhalation spray, nasal, vaginal, rectal, sublingual, or topical routes and may be formulated, alone or together, in suitable dosage unit formulations containing conventional non-toxic pharmaceutically acceptable carriers, adjuvants and vehicles appropriate for each route of administration.

A compound of the present disclosure may be used to treat animals, including mice, rats, horses, cattle, sheep, dogs, cats, and monkeys. However, a compound of the present disclosure may also be used in other organisms, such as avian species (e.g., chickens). One or more of the compounds of the present disclosure may also be effective for use in humans. The term “subject” or alternatively referred to herein as “patient” is intended to be referred to an animal, such as a mammal, for example a human, who has been the object of treatment, observation or experiment. However, one or more of the compounds, methods and pharmaceutical compositions of the present disclosure may be used in the treatment of animals. Accordingly, as used herein, a “subject” may be a human, non-human primate, rat, mouse, cow, horse, pig, sheep, goat, dog, cat, etc. The subject may be suspected of having or at risk for having a condition that may require inhibition of the inactivation of a Naᵥ1.5 voltage-gated sodium channel.

An “effective amount” of a compound according to the present disclosure may include a therapeutically effective amount or a prophylactically effective amount. A “therapeutically effective amount” refers to an amount effective, at dosages and for periods of time necessary, to achieve the desired therapeutic result, such as inhibition of the inactivation of a Naᵥ1.5 voltage-gated sodium channel. A therapeutically effective amount of a compound may vary according to factors such as the disease state, age, sex, and weight of the individual, and the ability of the compound to elicit a desired response in the individual. Dosage regimens may be adjusted to provide the optimum therapeutic response. A therapeutically effective amount may also be one in which any toxic or detrimental effects of the compound are outweighed by the therapeutically beneficial effects. A “prophylactically effective amount” may refer to an amount effective, at dosages and for periods of time necessary, to achieve the desired prophylactic result, such as inhibition of the inactivation of a Naᵥ1.5 voltage-gated sodium channel. Typically, a prophylactic dose may be used in subjects prior to or at an earlier stage of disease, so that a prophylactically effective amount may be less than a therapeutically effective amount. A suitable range for therapeutically or prophylactically effective amounts of a compound may be any integer from 0.1 nM-0.1 M, 0.1 nM-0.05 M, 0.05 nM-15 μM or 0.01 nM-10 μM.

In alternative embodiments, in the treatment or prevention of conditions which may require inhibition of the inactivation of a Naᵥ1.5 voltage-gated sodium channel, an appropriate dosage level may generally be about 0.01 to 500 mg per kg subject body weight per day and may be administered in single or multiple doses. In some embodiments, the dosage level may be about 0.1 to about 250 mg/kg per day. It will be understood that the specific dose level and frequency of dosage for any particular patient may be varied and may depend upon a variety of factors including the activity of the specific compound used, the metabolic stability and length of action of that compound, the age, body weight, general health, sex, diet, mode and time of administration, rate of excretion, drug combination, the severity of the particular condition, and the patient undergoing therapy.

It is to be noted that dosage values may vary with the severity of the condition to be alleviated. For any particular subject, specific dosage regimens may be adjusted over time according to the individual need and the professional judgement of the person administering or supervising the administration of the compositions. Dosage ranges set forth herein are exemplary only and do not limit the dosage ranges that may be selected by medical practitioners. The amount of active compound(s) in the composition may vary according to factors such as the disease state, age, sex, and weight of the subject. Dosage regimens may be adjusted to provide the optimum therapeutic response. For example, a single bolus may be administered, several divided doses may be administered over time or the dose may be proportionally reduced or increased as indicated by the exigencies of the therapeutic situation. It may be advantageous to formulate parenteral compositions in dosage unit form for ease of administration and uniformity of dosage. In general, compounds of the present disclosure should be used without causing substantial toxicity, and as described herein, one or more of the compounds may exhibit a suitable safety profile for therapeutic use. Toxicity of a compound of the present disclosure may be determined using standard techniques, for example, by testing in cell cultures or experimental animals and determining the therapeutic index. In some circumstances however, such as in severe disease conditions, it may be necessary to administer substantial excesses of the compositions.

The present invention will be further illustrated in the following examples.

### EXAMPLES

Homology Modeling and Autodocking

Homology modeling was performed using the Swiss-Model server (swissmodel[dot]expasy[dot]org ³⁶. The newly cryo-EM solved American cockroach voltage-gated sodium channel (NaᵥPas) structure (3.8-Å resolution) and the pre-activated bacterial crystal structure of NaᵥAb were used as templates against the Naᵥ1.5 sequence. PyMOL-pdb viewer was used for optimization and structure visualization.

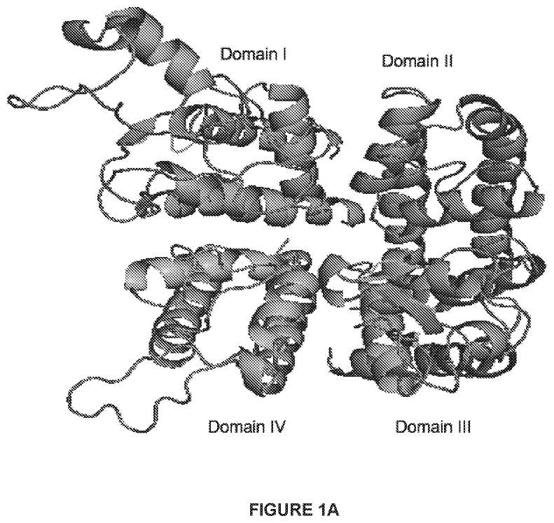

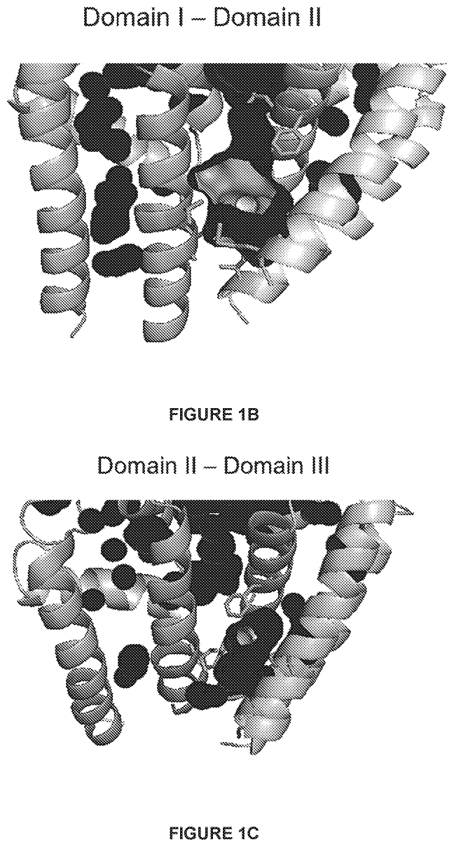

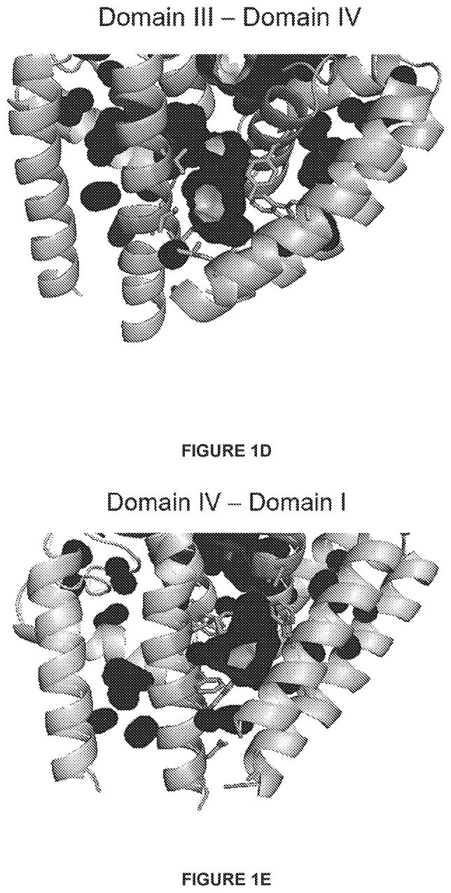

FIGS. 1A-E show the fenestrations from the exterior of NaᵥPas-Naᵥ1.5. The Naᵥ1.5 homology model based on NaᵥPas (NaᵥPas-Naᵥ1.5) indicated that the fenestrations between all domains in the homology model are relatively dilated, externally. However, their radial size substantially declines until a total obstruction is reached at the central cavity, except in Domain I-II. Residues outlining the fenestrations are included in Table 1. The residue numbers refer to the sequence set forth in SEQ ID NO: 1.

TABLE 1
Residues outlining fenestrations in NaᵥPas−Naᵥ 1.5 homology model

| Fenestrations | Residues |
|---|---|
| Domain I-II | M254, F362, L363, F366, V405, I408, V924, L928 |
| Domain II-III | L846, F892, F934, D1370, I1455, F1459 |
| Domain III-IV | L1338, W1345, Y1409, L1413, T1753, I1757, L1761 |
| Domain IV-I | W374, M394, F398, F402, N406, F1665, M1701, |
| | F1705, F1760, Y1767, I1768 |

Virtual docking was performed against the NaᵥAb-Naᵥ1.5 model, which adopts a structure closer to the ‘slow inactivated’ state in mammalian sodium channels as its crystal structure was captured at depolarized potentials ¹². The Naᵥ1.5 homology model built on NaᵥAb (Naᵥ1.5-NaᵥAb) was docked against the ZINC free database using DOCK Blaster server (blaster[dot]docking[dot]org, ³⁹). The highest 21 hits (Table 2) selected based on their binding affinity (kcal/mol) to Naᵥ1.5-NaᵥAb, were then virtually screened against Naᵥ1.5-NaᵥAb using AutoDock4 ³⁹. PyMOL-pdb viewer was used for optimization and visualization of the auto-docking results.

TABLE 2
Virtually Screened Compounds

| | | | xlogP | | | |
|---|---|---|---|---|---|---|
| ZINC | | | (pH | MW_ | Charge | tPSA |
| ID | Chemical Name | Chemical Structure | 7) | pH 7 | _pH 7 | (Å²) |
| ZINC 1232 3863 | N-(1H-benzimidazol- 2-yl)-4-methyl-2- pyrrol-1-yl-thiazole-5- carboxamide | 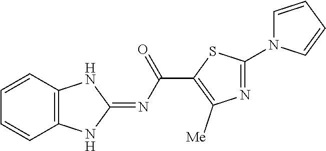 [SMILES: Cc1nc(-n2cccc2)sc1C(=O)N=c1[nH]c2ccccc2[nH]1] | 3.01 | 323.38 | 0 | 79 |
| ZINC 3853 2069 | N-(4-ethylphenyl)-2- oxo-benzimidazole-5- sulfonamide | 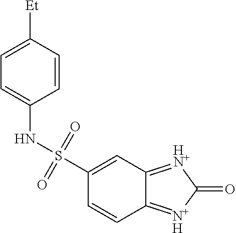 | 2.7 | 317.37 | 2 | 92 |
| ZINC 3876 7171 | N-(2-furylmethyl)-2,3- dioxo-quinoxaline-6- carboxamide | 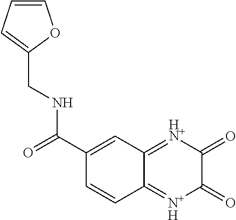 | 0.43 | 285.259 | 2 | 105 |
| ZINC 3876 7647 | N-(3-imidazol-1- ylpropyl)-2,3-dioxo- quinoxaline-6- carboxamide | 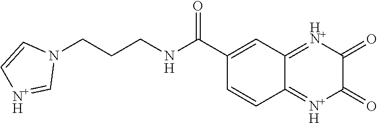 | −0.28 | 314.325 | 3 | 111 |
| ZINC 3877 6626 | N-(2-cyclohexen-1- ylethyl)-2,3-dioxo- quinoxaline-6- carboxamide | 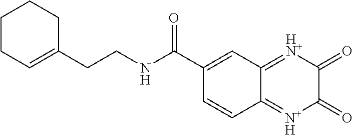 | 2.15 | 313.357 | 2 | 92 |
| ZINC 3941 1748 | 6-(4-methylpiperazin- 1- yl)sulfonylquinoxaline- 2,3-dione | 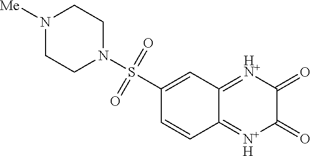 | −0.22 | 324.362 | 2 | 103 |
| ZINC 3969 9427 | N-allyl-2-oxo- benzimidazole-5- sulfonamide | 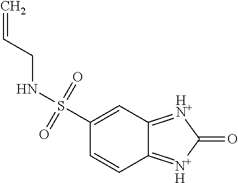 | 0.73 | 253.283 | 2 | 92 |
| ZINC 3970 9283 | N-(m-tolyl)-2,3-dioxo- quinoxaline-6- sulfonamide | 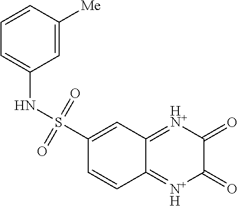 | 1.77 | 331.353 | 2 | 109 |
| ZINC 3972 9455 | N-(3-ethylphenyl)-2- oxo-benzimidazole-5- sulfonamide | 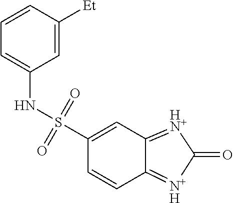 | 2.68 | 317.37 | 2 | 92 |
| ZINC 3977 6444 | N-ethyl-N-(4- fluorophenyl)-2-oxo- benzimidazole-5- sulfonamide | 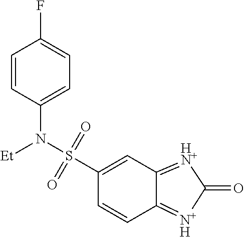 | 2.57 | 335.36 | 2 | 83 |
| ZINC 4001 4265 | N-[(2S)-2- (dimethylamino)-2-(2- furyl)ethyl]-2,3-dioxo- quinoxaline-6- carboxamide | 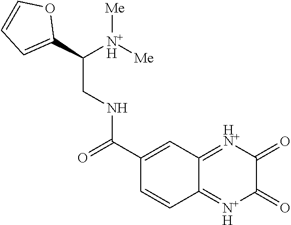 | 0.64 | 343.363 | 3 | 109 |
| ZINC 4001 4267 | N-[(2R)-2- (dimethylamino)-2-(2- furyl)ethyl]-2,3-dioxo- quinoxaline-6- carboxamide | 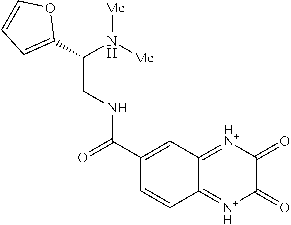 | 0,64 | 343.363 | 3 | 109 |
| ZINC 6390 8937 | N-[2-(4- methoxyphenyl)ethyl]- 2-oxo-benzimidazole- 5-sulfonamide | 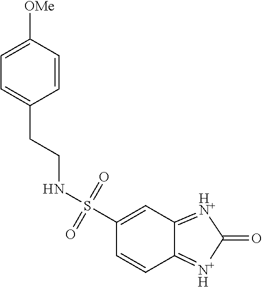 | 1.95 | 347.396 | 2 | 101 |
| ZINC 6397 0468 | N-methyl-N-[(2- methyl-3- furyl)methyl]-2-oxo- benzimidazole-5- sulfonamide | 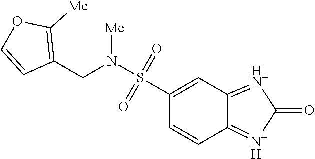 | 0.9 | 321.358 | 2 | 96 |
| ZINC 6400 2748 | 5-[2-[2- furylmethyl(methyl)- amino]acetyl]benzimid- azol-2-one | 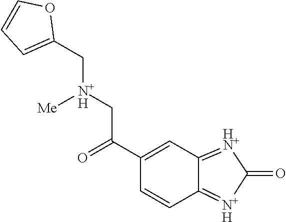 | 1.23 | 286.311 | 3 | 80 |
| ZINC 6401 2417 | N-(1-methylpyrazol-3- yl)-2,3-dioxo- quinoxaline-6- sulfonamide | 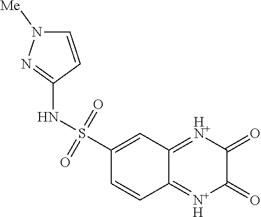 | −0.08 | 321.318 | 2 | 126 |
| ZINC 6447 0706 | N-(2-chloro-4-methyl- phenyl)-2-oxo- benzimidazole-5- sulfonamide | 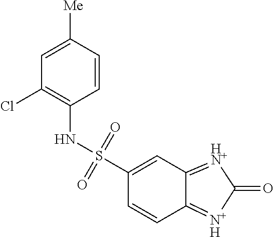 | 2.84 | 337.788 | 2 | 92 |
| ZINC 6447 0729 | N-(3-methoxyphenyl)- 2-oxo-benzimidazole- 5-sulfonamide | 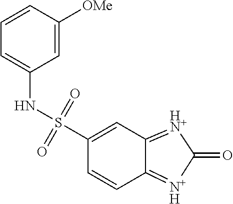 | 1.82 | 319.342 | 2 | 101 |
| ZINC 6447 0737 | N-(2-ethylphenyl)-2- oxo-benzimidazole-5- sulfonamide | 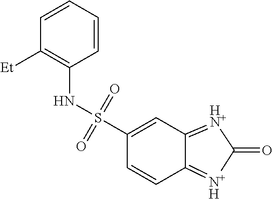 | 2.65 | 317.37 | 2 | 92 |
| ZINC 6447 0745 | N-(4-fluoro-2-methyl- phenyl)-2-oxo- benzimidazole-5- sulfonamide | 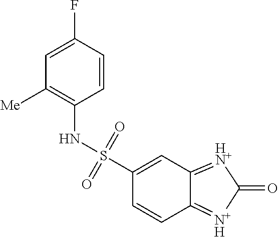 | 2.33 | 321.333 | 2 | 92 |
| ZINC 6447 0747 | N-[(2- methoxyphenyl)methyl]- 2-oxo- benzimidazole-5- sulfonamide | 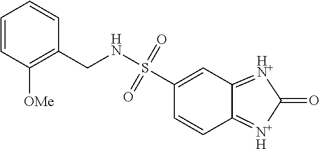 | 1.5 | 333.369 | 2 | 101 |

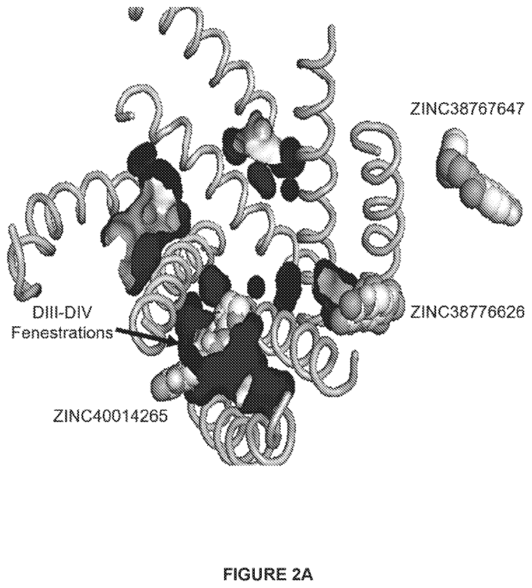

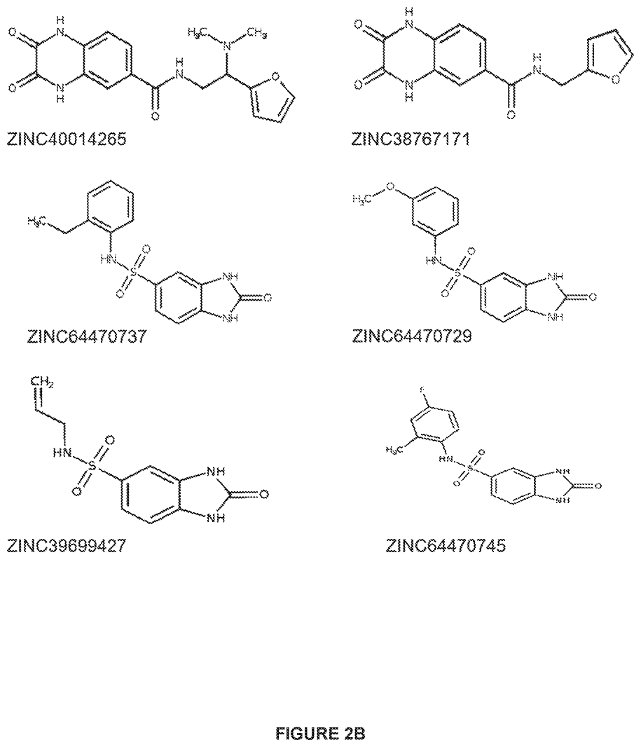

The compounds can be generally categorized into carboxamides and sulfonamides. These compounds were auto-docked against NaᵥAb-Naᵥ1.5 using AutoDock4. Six hits, namely compounds ZINC38767171, ZINC39699427, ZINC40014265, ZINC64470729, ZINC64470737 and ZINC64470745 are shown in FIGS. 2A-B. Only three of those compounds are shown in their highest affinity binding mode to Naᵥ1.5. The compound, ZINC40014265, formed Van der Waals interaction within the Domain III-IV fenestration compared to ZINC38776626 which is partially suspended in Domain II-III fenestration. ZINC38767747 is free from any fenestration interaction and easily accesses the inner vestibule of the channel in other binding modes. Other compounds shown in FIG. 2B also bind to Domain III-IV fenestration.

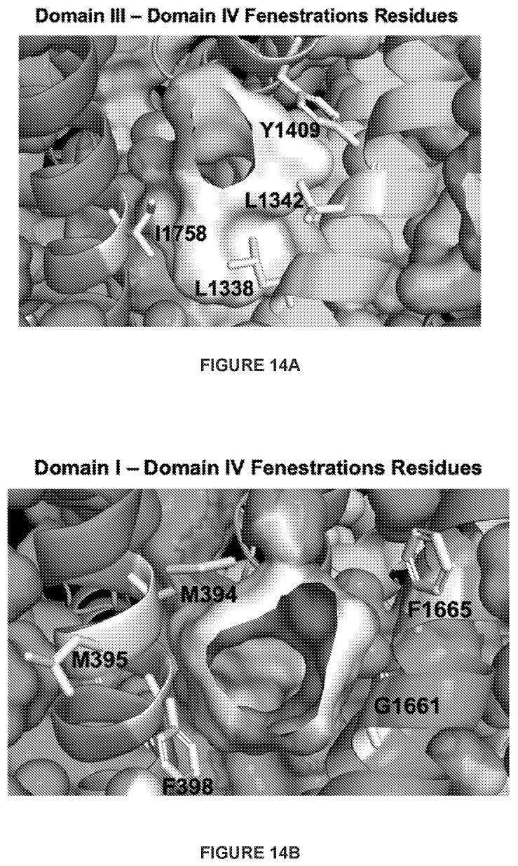

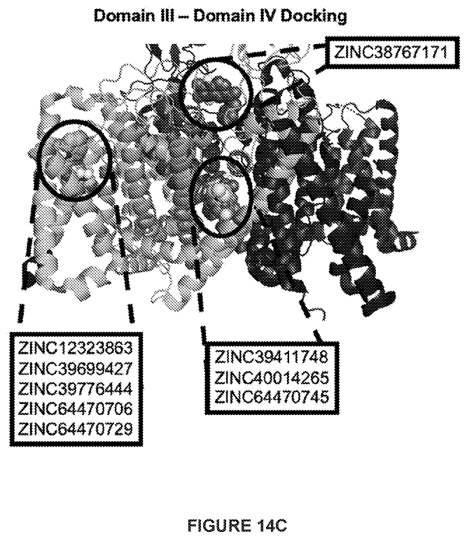

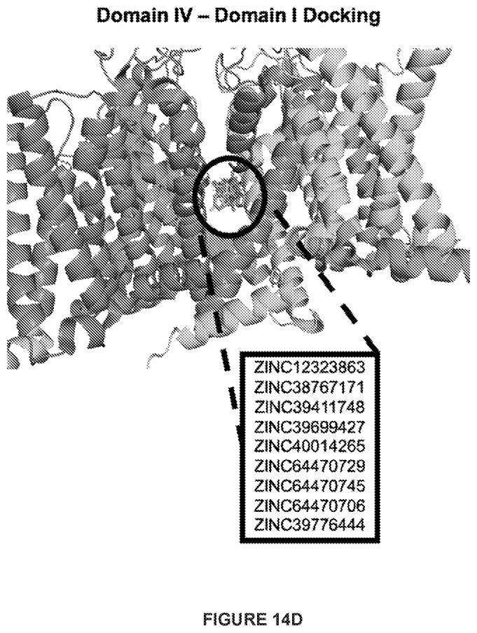

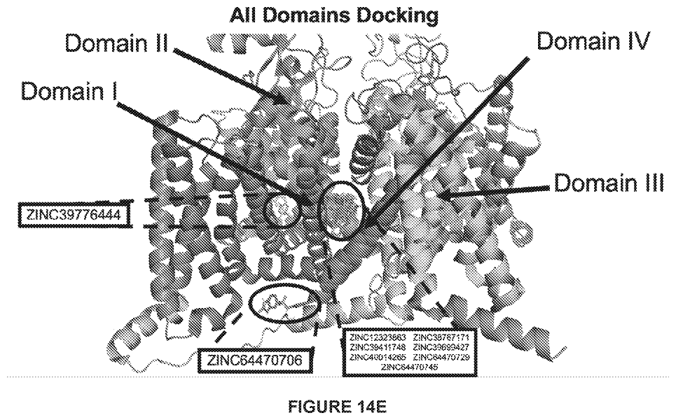

The compounds of Table 2 were also docked against the rNaᵥ1.5 channel ³². Compounds were screened against individual fenestrations: Domain I-Domain 11, Domain II-Domain III, Domain III-Domain IV, and Domain I-IV. Finally, the compounds were screened against the whole rNaᵥ1.5, with the four intact domains. The highest binding affinity mode with a rmsd=0 are reported in Table 6 and shown in FIGS. 14A-E.

TABLE 6
Binding sites and affinities for compounds docked against rNav1.5

| | Highest Affinity Binding Mode (kcal/mol) | | | | | | | | | |
|---|---|---|---|---|---|---|---|---|---|---|
| Compounds | DI-DII | | DII-DIII | | DIII-DIV | | DIV-DI | | All Domains | |
| ZINC12323863 | −8.0 | VSD | −7.4 | OUT VES | −8.7 | VSD | −8.4 | FEN | −8.6 | DIV-DI FEN |
| ZINC38532069 | −7.9 | VSD | −8.0 | OUT VES | −8.5 | FEN | −8.5 | FEN | −8.9 | DIV-DI FEN |
| ZINC38767171 | −7.0 | VSD | −7.5 | VSD | −6.9 | OUT VES | −7.7 | FEN | −8.0 | DIV-DI FEN |
| ZINC38767647 | −7.9 | VSD | −7.9 | VSD | −7.2 | FEN | −7.8 | FEN | −7.6 | DIV-DI FEN |
| ZINC38776626 | −7.5 | VSD | −7.4 | OUT VES | −9.1 | FEN | −8.1 | FEN | −8.8 | DIV-DI FEN |
| ZINC39411748 | −8.0 | VSD | −7.0 | VSD | −7.6 | FEN | −8.4 | FEN | −8.5 | DIV-DI FEN |
| ZINC39699427 | −6.6 | VSD | −6.4 | VSD | −6.9 | VSD | −6.8 | FEN | −7.0 | DIV-DI FEN |
| ZINC39709283 | −8.5 | VSD | −7.9 | VSD | −8.9 | VSD | −9.0 | VSD | −9.3 | DIV-DI FEN |
| ZINC39729455 | −7.7 | VSD | −7.1 | VSD | −8.5 | FEN | −8.4 | FEN | −8.8 | DIV-DI FEN |
| ZINC39776444 | −7.7 | VSD | −7.1 | VSD | −7.5 | VSD | −8.4 | FEN | −7.8 | VSDI |
| ZINC40014265 | −8.3 | VSD | −7.4 | OUT VES | −7.7 | FEN | −8.0 | FEN | −8.6 | DIV-DI FEN |
| ZINC40014267 | −8.3 | VSD | −7.2 | OUT VES | −7.3 | FEN | −7.7 | FEN | −8.7 | DIV-DI FEN |
| ZINC63908937 | −8.7 | VSD | −7.8 | VSD | −8.5 | FEN | −8.0 | FEN | −8.5 | DIV-DI FEN |
| ZINC63970468 | −7.2 | VSD | −7.0 | INN VES | −7.3 | FEN | −8.2 | FEN | −8.5 | DIV-DI FEN |
| ZINC64002748 | −6.6 | VSD | −6.8 | VSD | −7.1 | FEN | −7.5 | FEN | −7.5 | VSDI |
| ZINC64012417 | −8.0 | VSD | −7.1 | OUT VES | −8.4 | VSD | −8.2 | FEN | −8.2 | DIV-DI FEN |
| ZINC64470706 | −9.3 | VSD | −7.0 | VSD | −8.0 | VSD | −8.2 | FEN | −7.0 | DIII S4-S5 |
| ZINC64470729 | −7.3 | VSD | −7.3 | VSD | −7.9 | VSD | −8.6 | FEN | −9.0 | DIV-DI FEN |
| ZINC64470737 | −7.6 | VSD | −7.5 | VSD | −8.7 | FEN | −8.1 | FEN | −8.8 | DIV-DI FEN |
| ZINC64470745 | −8.1 | VSD | −7.9 | VSD | −8.7 | FEN | −8.8 | FEN | −9.0 | DIV-DI FEN |
| ZINC64470747 | −7.8 | VSD | −7.2 | OUT VES | −7.5 | FEN | −8.3 | FEN | −8.5 | DIV-DI FEN |

*VSD = Voltage-Sensing Domain
*OUT VES = Outer Vestibule
*INN VES = Inner Vestibule
*FEN = Fenestration

None of the compounds tested in this screen interacted with Domain I-Domain II and Domain II-Domain III fenestrations (Table 6). The tested compounds had a high affinity, however, for Domain III-IV and Domain I-IV fenestrations (Table 6 and FIGS. 14A-E). The resides that outline the outer diameter of these two fenestrations are shown in FIGS. 14A-B. When docked against Domain III-IV fenestrations only, ZINC40014265 and ZINC64470745 along with ZINC39411748 bind within the fenestrations as opposed to other compounds (FIG. 14C) indicating that these compounds inhibit progression of inactivation in Naᵥ1.5 by interacting with the fenestrations. Compounds that were shown to block peak sodium current (by whole-cell patch clamp technique with single voltage-pulse protocol) such as ZINC38767171 bind to the outer vestibule. All compounds screened using high-throughput patch clamp techniques and double voltage-pulse protocol interacted with Domain IV-I fenestration in rNaᵥ1.5 (FIG. 14D). When all four domains were reannealed together to form an intact channel, most compounds interacted with Domain IV-I fenestrations (FIG. 14E) compared to Domain III-IV fenestrations (FIG. 14C).

Electrophysiology

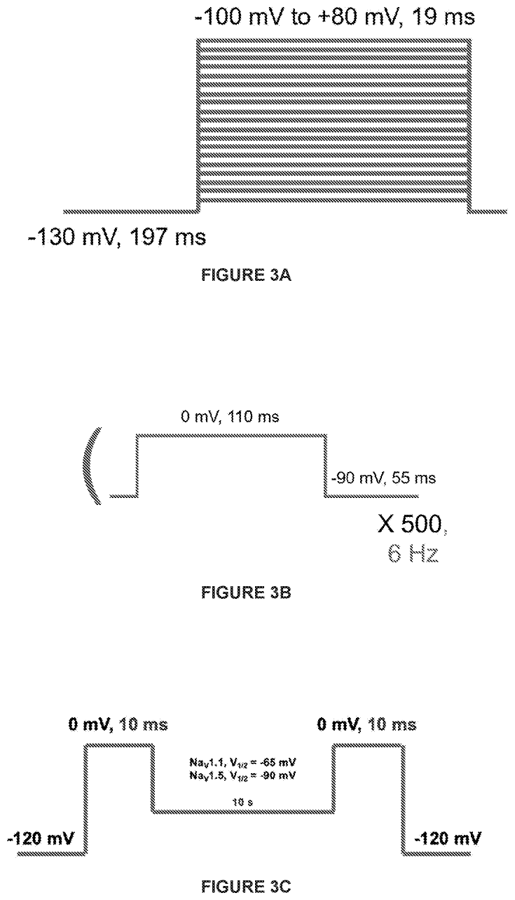

In one set of studies, we used a whole-cell patch clamp technique with single voltage-pulse protocols (FIGS. 3A-B) to characterize compounds ZINC38767171, ZINC39699427, ZINC40014265, ZINC64470729, ZINC64470737 and ZINC64470745. Whole-cell patch clamp was performed in extracellular solution containing (in mM): 140 NaCl, 4 KCl, 2 CaCl₂, 1 MgCl₂, and 10 HEPES (pH 7.4 adjusted with CsOH). The intracellular pipettes were dipped in dental wax and thermally polished to attain a resistance of 1.0-1.5 MΩ. The intracellular solutions contained (in mM): 130 CsF, 10 NaCl, 10 HEPES, and 10 EGTA titrated to pH 7.4. The six compounds, ZINC38767171, ZINC39699427, ZINC40014265, ZINC64470729, ZINC64470737 and ZINC64470745, chosen for electrophysiology characterization, ordered from MolPort (Easy compound ordering services), were diluted to 10 μM, 50 μM, and 100 μM in extracellular solution for biophysical assessment of channel gating.

The six compounds were screened against wild type (WT) Naᵥ1.5-α subunits at the optimal physiological temperature of 37° C. Human Embryonic Kidney (HEK293) cells were transfected with WT Naᵥ1.5-α subunits (transient transfections). The α subunits were co-expressed with the β1 subunit and eGFP (enhanced green fluorescence protein) in a 2:1:2 ratio, respectively.

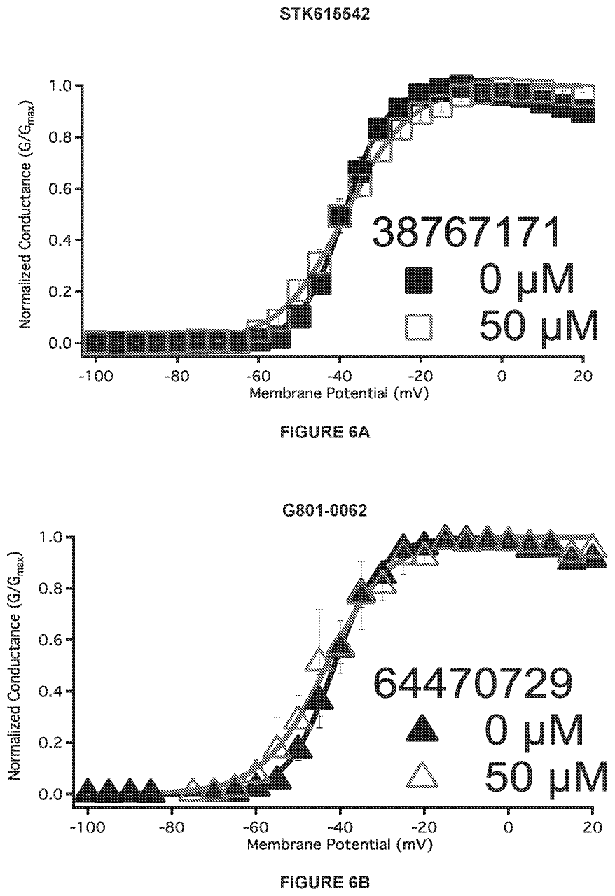

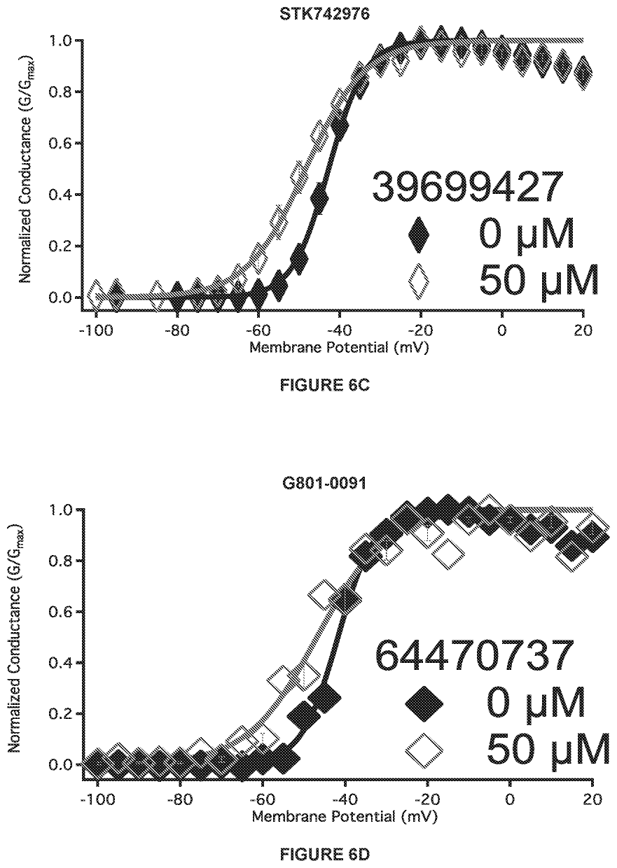

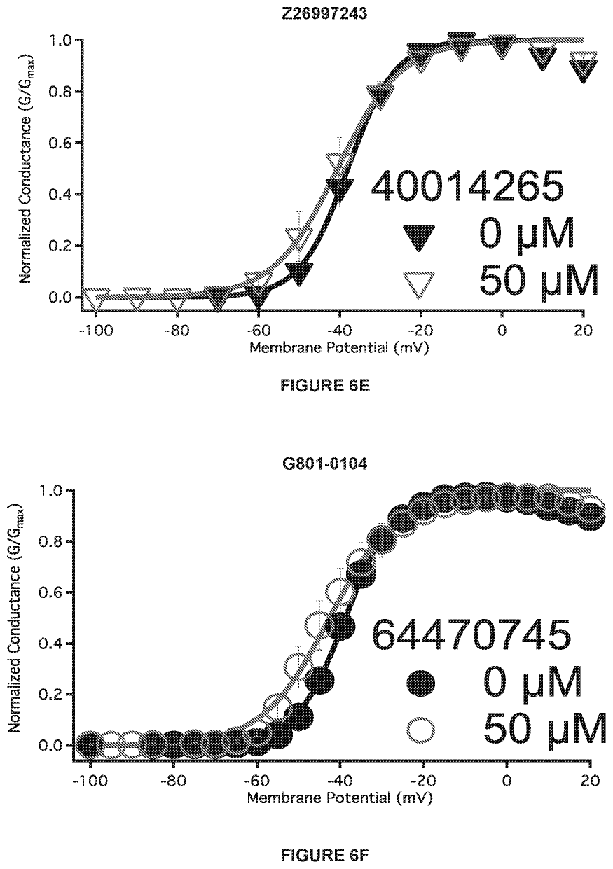

Conductance was measured by a protocol that depolarized the membrane from −100 mV to +80 mV in increments of +5 mV for 19 ms. Prior to the test pulse, channels were allowed to recover from fast inactivation at −130 mV for 197 ms. The tested compounds had no effect on the conductance midpoint (FIGS. 6A-F).

At voltages greater than −50 mV, the fast inactivation T values were calculated from the mono-exponential fit to the decay of sodium current.

Slow inactivation (SI) was indirectly determined by measuring use-dependence, which is a physiologically-relevant protocol. The protocol includes a series of 500 110 ms depolarizing pulses to 0 mV followed by a 55 ms-90 mV recovery pulse at a frequency 6 Hz. With repetitive depolarizations, Naᵥ1.5 channels accumulate into slow inactivation.

TABLE 3
Compounds effects on peak I_{Na} (Single voltage-pulse protocol)

| Treatment | Peak INa −20 mV | % Block −20 mV |
|---|---|---|
| 38767171-0 | −0.95 ± 0.04 | 0.00 |
| 38767171-10 | −0.72 ± 0.06 | −24.21 |
| 38767171-50 | −0.59 ± 0.11 | −37.89 |
| 38767171-100 | −0.58 ± 0.10 | −38.95 |
| 39699427-0 | −1.00 ± 0.00 | 0.00 |
| 39699427-10 | −0.66 ± 0.04 | −34.00 |
| 39699427-50 | −0.58 ± 0.06 | −42.00 |
| 39699427-100 | −0.53 ± 0.07 | −47.00 |
| 40014265-0 | −1.00 ± 0.01 | 0.00 |
| 40014265-10 | −0.80 ± 0.06 | −20.00 |
| 40014265-50 | −0.63 ± 0.06 | −37.00 |
| 40014265-100 | −0.59 ± 0.06 | −41.00 |
| 64470729-0 | −0.97 ± 0.02 | 0.00 |
| 64470729-10 | −0.75 ± 0.05 | −22.68 |
| 64470729-50 | −0.72 ± 0.06 | −25.77 |
| 64470729-100 | −0.60 ± 0.26 | −38.14 |
| 64470737-0 | −0.98 ± 0.01 | 0.00 |
| 64470737-10 | −0.76 ± 0.13 | −22.45 |
| 64470737-50 | −0.58 ± 0.10 | −40.82 |
| 64470737-100 | −0.25 ± 0.00 | −74.49 |
| 64470745-0 | −0.97 ± 0.01 | 0.00 |
| 64470745-10 | −0.74 ± 0.06 | −23.71 |
| 64470745-50 | −0.85 ± 0.08 | −12.37 |
| 64470745-100 | −0.73 ± 0.04 | −24.74 |

TABLE 4
Peak I_{Na} Block and UDI Block Hill Curve IC₅₀ and Slope (Single voltage−
pulse protocol)

| | Peak INa | | UDI Block | |
|---|---|---|---|---|
| Treatment | IC50 (μM) | Slope | IC50 (μM) | Slope |
| 38767171 | 7.611 | 1.697 | 0.251 | 3.161 |
| 39699427 | 6.451 | 0.236 | 0.196 | 4.556 |
| 40014265 | 12.56 | 1.064 | 268.58 | 0.511 |
| 64470729 | 34.63 | 0.389 | 40.73 | −0.194 |
| 64470737 | 9.862 | 14.37 | 95.10 | 0.710 |
| 64470745 | 0.928 | 16.06 | 901.26 | 0.289 |

Table 4 shows the IC₅₀ values and slope (rate) of the Hill curves for peak I_{Na} and UDI block by six compounds tested using the single voltage-pulse protocols. The aliphatic sulfonamide, ZINC39699427, had the lowest IC₅₀ (indicative of inhibition) for both peak I_{Na} and UDI block. The drugs that blocked peak I_{Na} with the least affinity (indicative of potentiation) at rest were ZINC40014265 and ZINC64470729. The drugs that blocked use-dependence the least were ZINC40014265 and ZINC6470745.

TABLE 5
Compounds effects on fast inactivation onset time constant (ms)
(Single voltage−pulse protocol)

| Treatment | FI −50 mV | FI −30 mV | FI −10 mV | FI +10 mV |
|---|---|---|---|---|
| 38767171-0 | 2.45 ± 0.30 | 0.68 ± 0.07 | 0.39 ± 0.06 | 0.26 ± 0.05 |
| 38767171-10 | 1.70 ± 0.33 | 0.71 ± 0.12 | 0.39 ± 0.06 | 0.25 ± 0.05 |
| 38767171-50 | 1.79 ± 0.15 | 0.94 ± 0.10 | 0.58 ± 0.08 | 0.34 ± 0.06 |
| 38767171-100 | 1.92 ± 0.38 | 1.05 ± 0.21 | 0.60 ± 0.13 | 0.41 ± 0.11 |
| 39699427-0 | 1.82 ± 0.23 | 0.48 ± 0.07 | 0.29 ± 0.05 | 0.21 ± 0.04 |
| 39699427-10 | 1.32 ± 0.24 | 0.54 ± 0.09 | 0.31 ± 0.04 | 0.21 ± 0.03 |
| 39699427-50 | 1.46 ± 0.09 | 0.63 ± 0.08 | 0.35 ± 0.04 | 0.25 ± 0.03 |
| 39699427-100 | 1.61 ± 0.18 | 0.72 ± 0.04 | 0.40 ± 0.03 | 0.26 ± 0.02 |
| 40014265-0 | 3.26 ± 0.96 | 0.54 ± 0.08 | 0.27 ± 0.03 | 0.19 ± 0.02 |
| 40014265-10 | 2.98 ± 0.84 | 0.63 ± 0.07 | 0.33 ± 0.03 | 0.23 ± 0.02 |
| 40014265-50 | 2.18 ± 0.70 | 0.69 ± 0.11 | 0.37 ± 0.05 | 0.25 ± 0.03 |
| 40014265-100 | 2.17 ± 0.73 | 0.69 ± 0.17 | 0.37 ± 0.09 | 0.26 ± 0.07 |
| 64470729-0 | 2.24 ± 0.46 | 0.54 ± 0.05 | 0.29 ± 0.02 | 0.20 ± 0.02 |
| 64470729-10 | 1.67 ± 0.21 | 0.70 ± 0.14 | 0.40 ± 0.08 | 0.26 ± 0.04 |
| 64470729-50 | 1.97 ± 0.29 | 0.75 ± 0.07 | 0.40 ± 0.03 | 0.26 ± 0.02 |
| 64470729-100 | 1.71 ± 0.22 | 0.76 ± 0.09 | 0.39 ± 0.02 | 0.23 ± 0.03 |
| 64470737-0 | 1.74 ± 0.31 | 0.45 ± 0.05 | 0.27 ± 0.03 | 0.20 ± 0.02 |
| 64470737-10 | 1.37 ± 0.31 | 0.66 ± 0.09 | 0.41 ± 0.07 | 0.31 ± 0.06 |
| 64470737-50 | 1.88 ± 0.27 | 1.02 ± 0.13 | 0.64 ± 0.08 | 0.39 ± 0.02 |
| 64470737-100 | 1.82 ± 0.00 | 0.96 ± 0.00 | 0.66 ± 0.00 | 0.49 ± 0.00 |
| 64470745-0 | 2.67 ± 0.38 | 0.52 ± 0.04 | 0.27 ± 0.02 | 0.19 ± 0.02 |
| 64470745-10 | 2.12 ± 0.34 | 0.66 ± 0.04 | 0.37 ± 0.03 | 0.27 ± 0.03 |
| 64470745-50 | 1.75 ± 0.34 | 0.63 ± 0.05 | 0.37 ± 0.03 | 0.25 ± 0.02 |
| 64470745-100 | 1.79 ± 0.20 | 0.81 ± 0.12 | 0.45 ± 0.07 | 0.28 ± 0.04 |

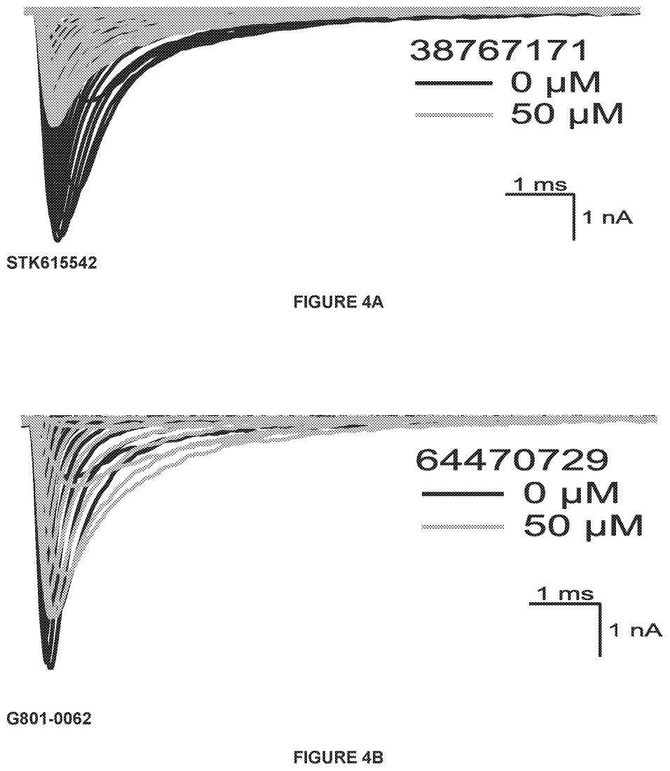

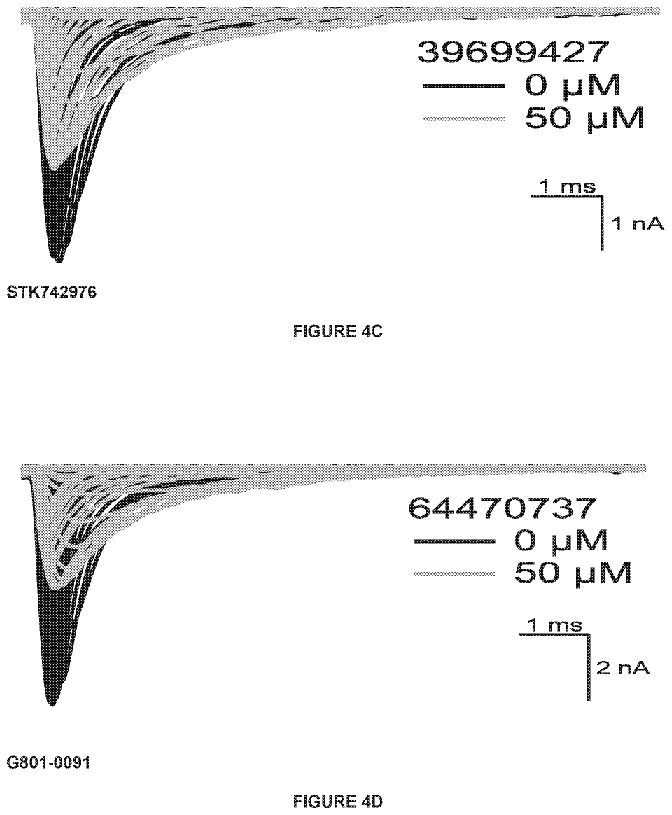

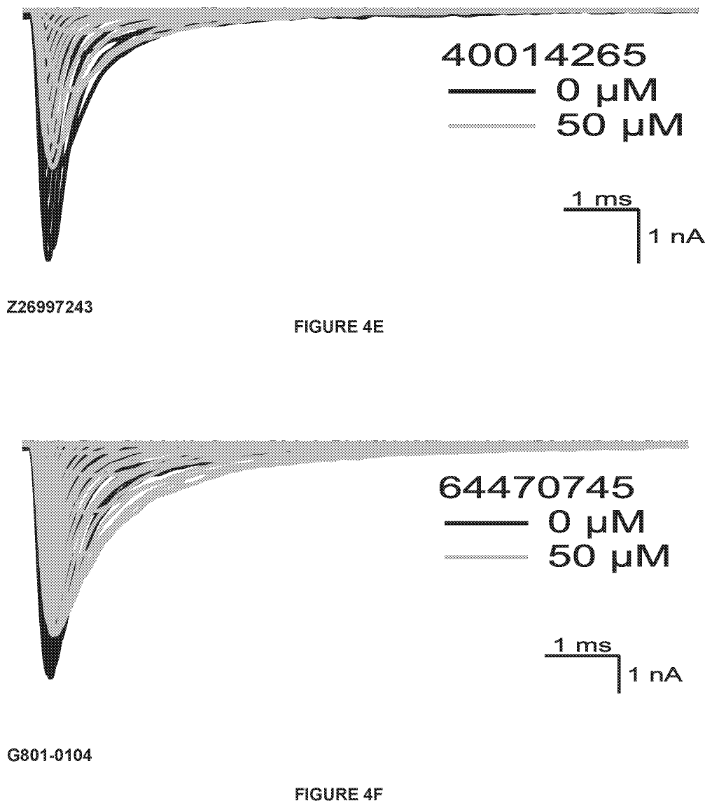

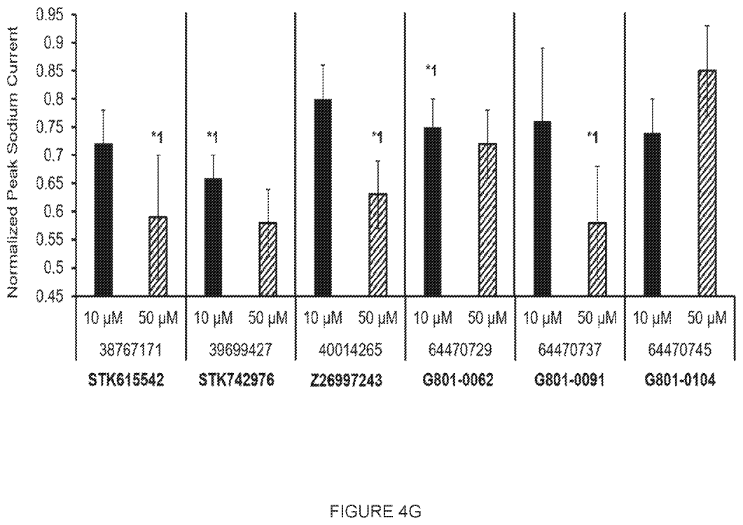

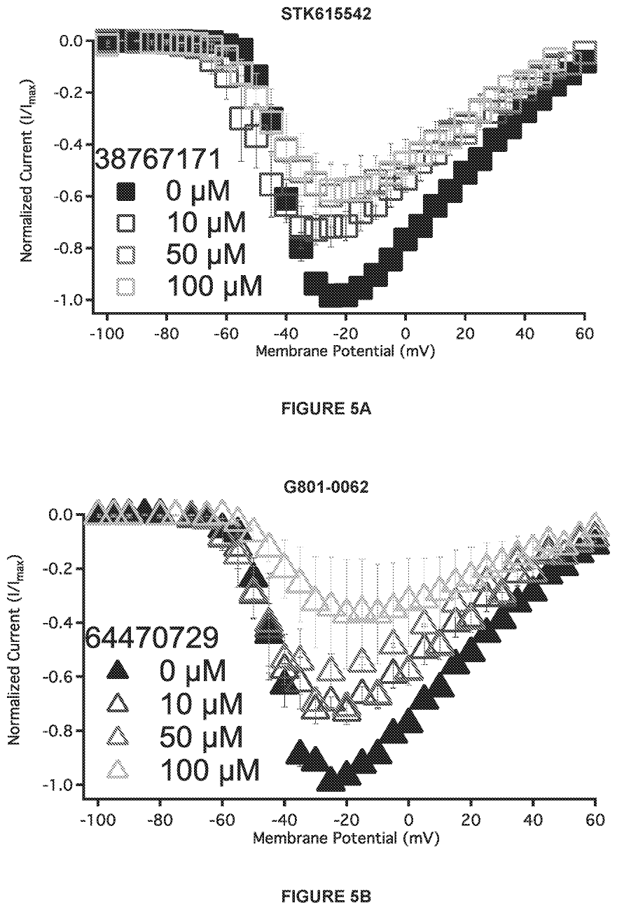

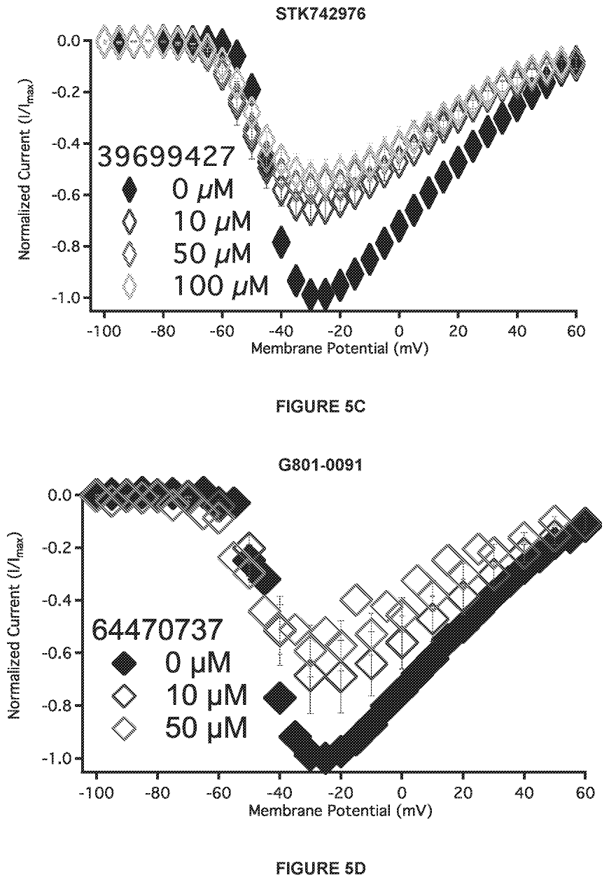

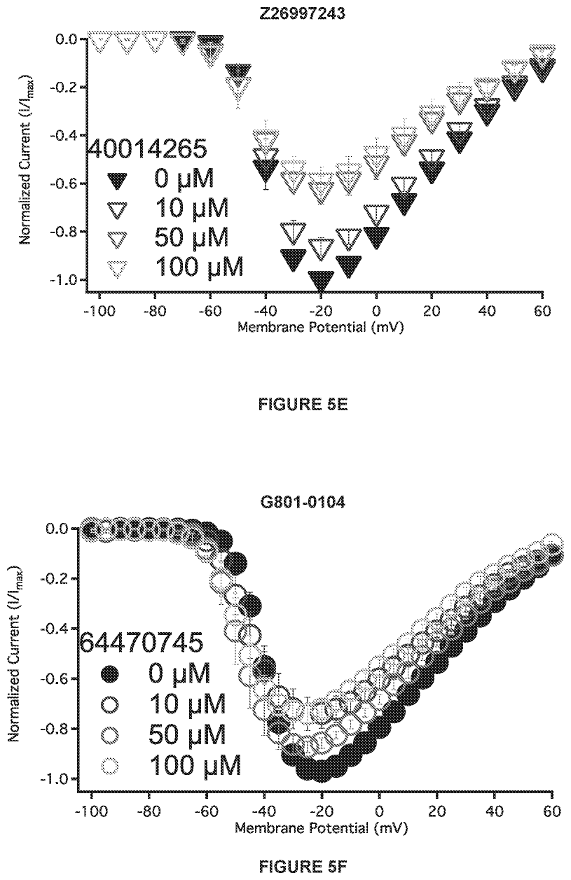

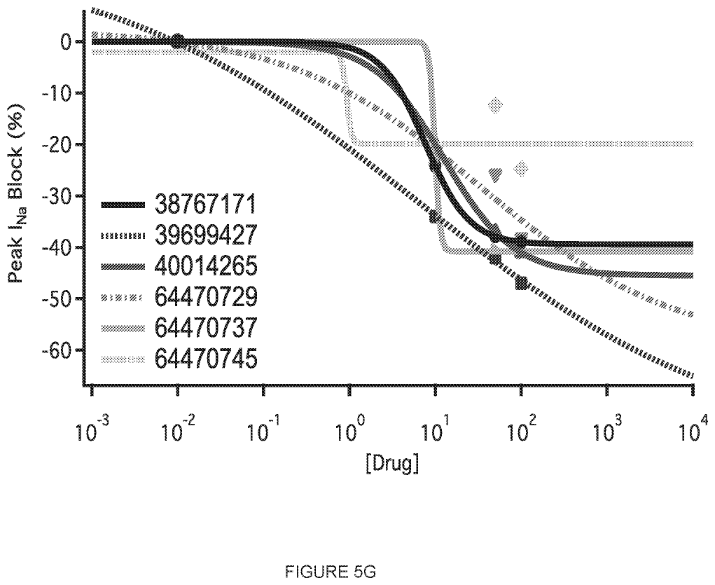

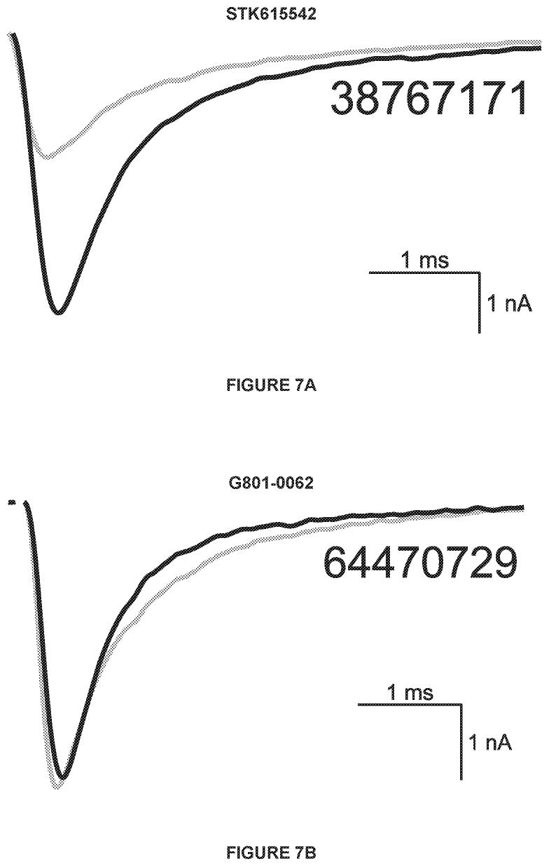

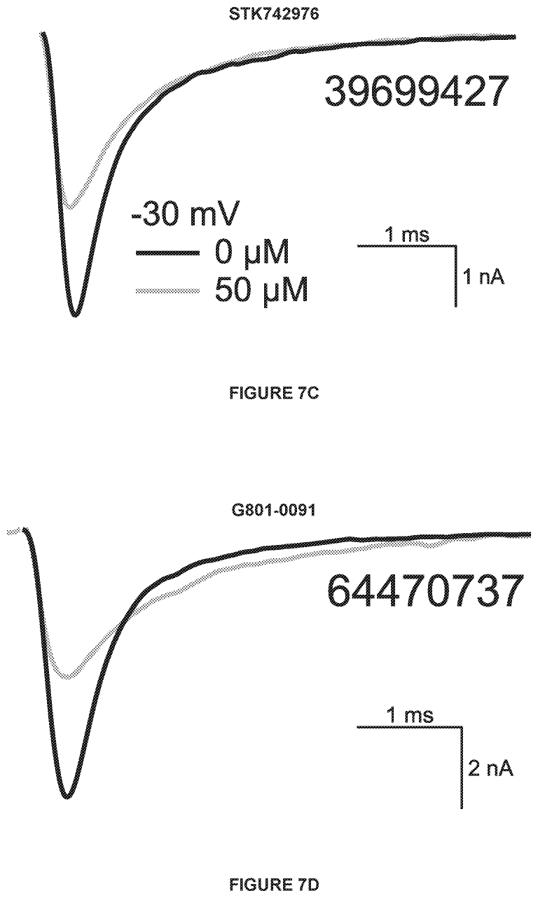

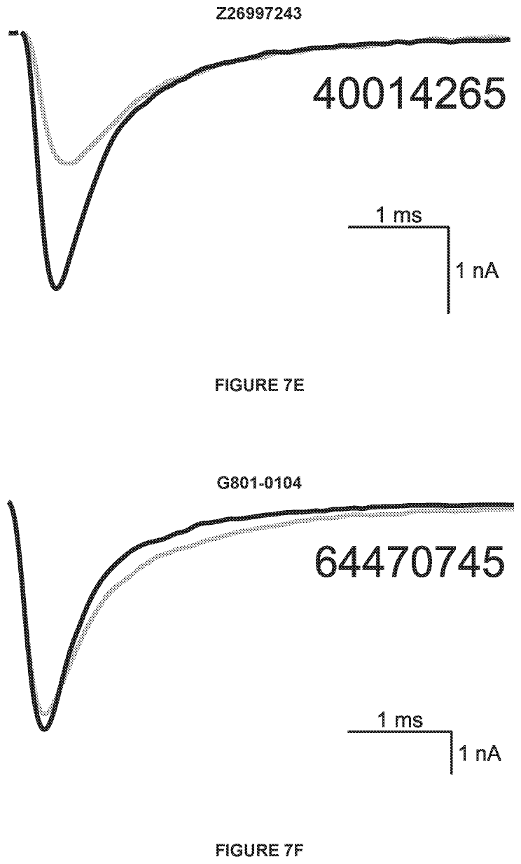

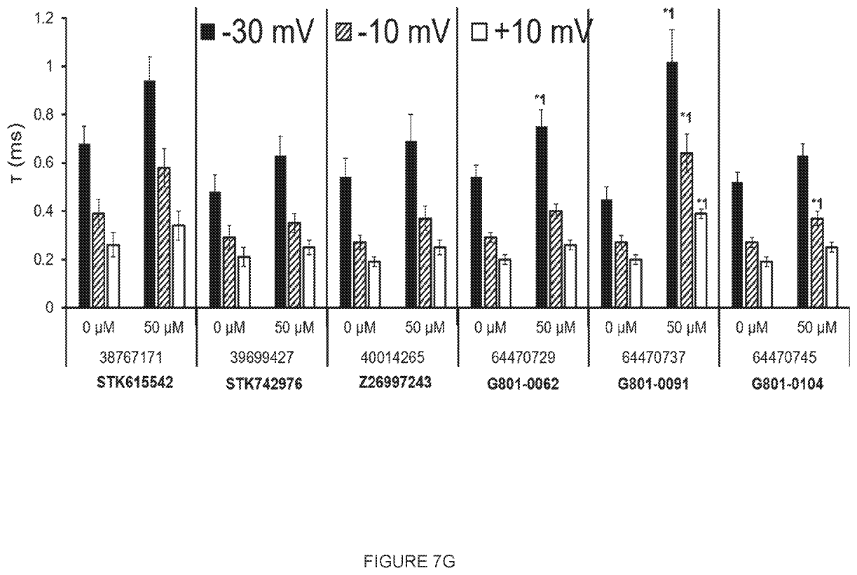

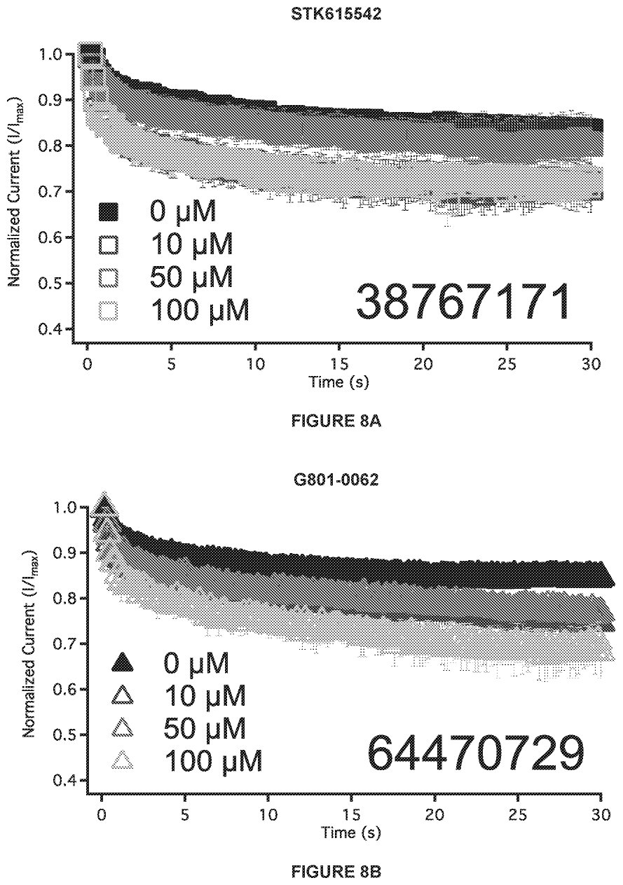

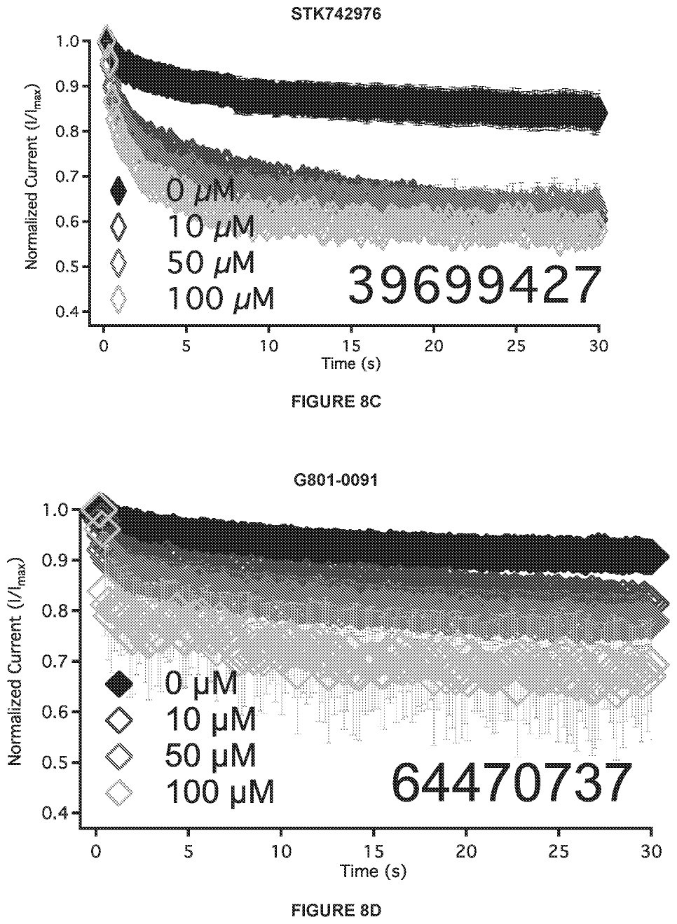

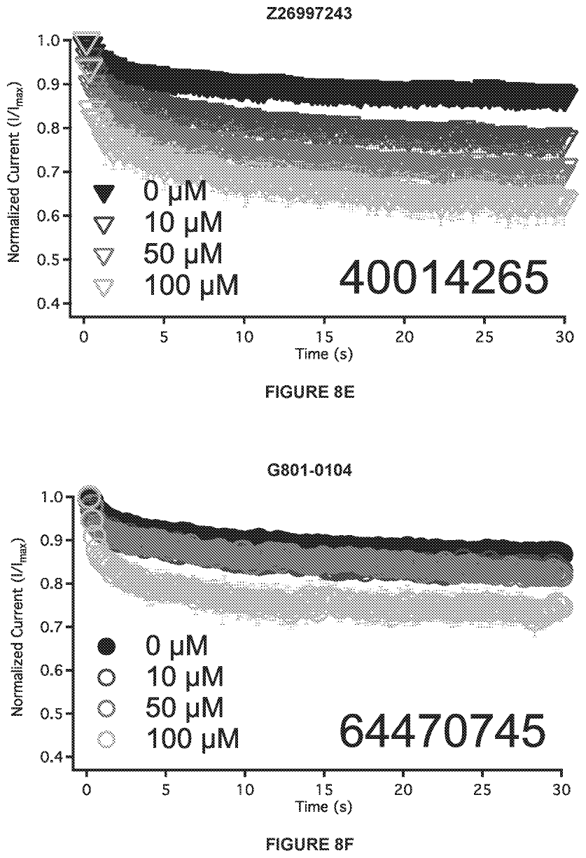

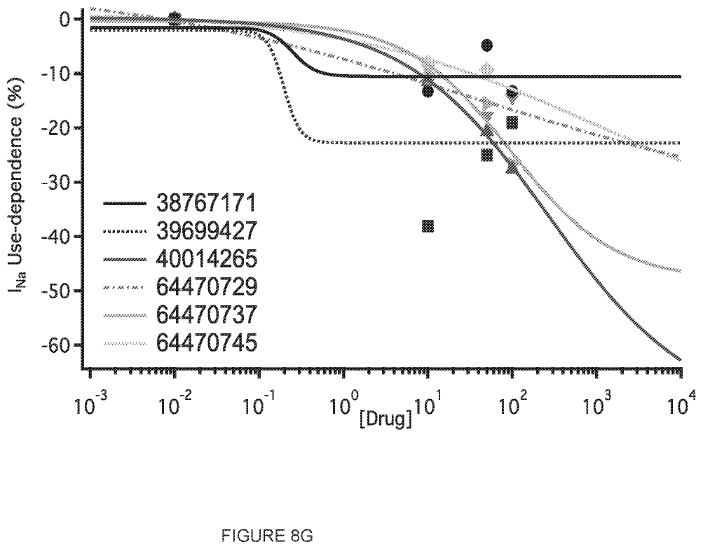

All six compounds characterized using whole-cell electrophysiology and single voltage pulse protocols had differential potencies for peak I_{Na} (FIGS. 4A-G, FIGS. 5A-G, FIGS. 6A-G, and Table 3). ZINC64470745 blocked peak I_{Na} the least compared to the other compounds. All of ZINC64470737, ZINC64470729, ZINC38767171, and ZINC64470745 significantly decelerated the fast inactivation kinetics at open-state voltages (between −30 and +10 mV, FIGS. 7A-G, Table 5). These compounds also affected the use-dependence inactivation the least (FIGS. 8A-G). ZINC39699427 significantly enhanced use-dependence in Naᵥ1.5 and also considerably blocked peak I_{Na} at low concentrations (FIGS. 8-G). Without being bound to any particular theory, the biophysical shifts differentiating between ZINC39699427 and the other compounds could be explained by the fact that ZINC39699427 has an aliphatic compared to an aromatic side group, which may suggest that the fenestrations in Naᵥ1.5 are resisted by an aromatic side-chain compared to an aliphatic chain which can easily traverse the lateral pores and settle in the central pore, blocking I_{Na}.

In another set of studies, we used high-throughput patch clamp techniques using the QPatch (Qube) 384 to screen compounds against both neuronal and cardiac sodium channels. We used a different electrophysiology assay (double voltage-pulse protocol; FIG. 3C) to detect potentiators, inhibitors, and compounds that do both. The protocol assesses these parameters at both rest and partially inactivated holding potentials. For this assay, we used nine compounds from Table 2 that were diluted to 50 μM in DMSO for biophysical assessment of channel gating. The extracellular solution used in these experiments was Ringer solution.

The nine compounds were screened against the wild type (WT) cardiac (Naᵥ1.5) and neuronal (Naᵥ1.1) sodium channel α-subunits stably expressed in Human Embryonic Kidney (HEK293) along with the β1-subunit.

Currents were assessed by a test pulse of 0 mV from a holding potential at rest (−120 mV) before preconditioning at the appropriate V_{1/2} for the sodium channel subtype (V_{1/2}=−90 mV and V_{1/2}=−65 mV for Naᵥ1.5 and Naᵥ1.1, respectively) for 10 s. Parameters were tested following 5 min of incubation with the compounds and normalized to control (vehicle). Peak current, the time constant of inactivation, and the area under the curve (AUC) were measured.

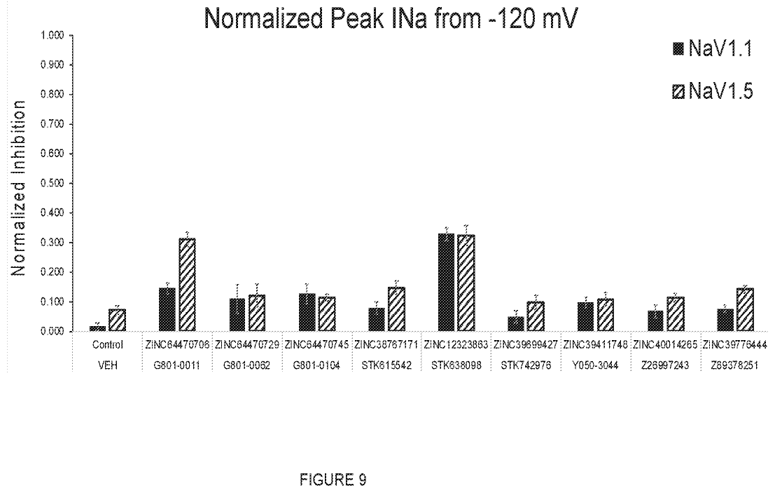

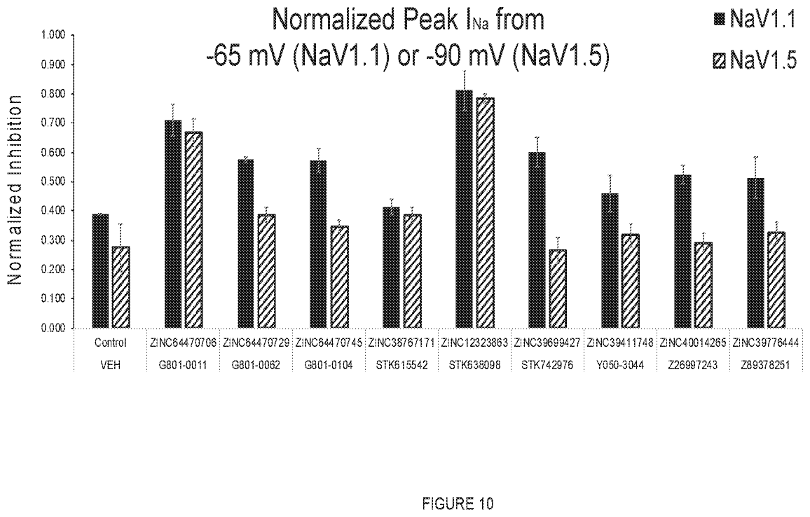

Normalized sodium current inhibition measured after holding the potential at −120 mV is shown as a function of the nine compounds tested at 50 μM in FIG. 9. ZINC12323863 inhibits peak sodium current non-selectively in both Naᵥ1.1 and Naᵥ1.5 by ˜32%. This effect is further exacerbated when normalized inhibition is measured after a 10 s pulse to mid-voltage potential: ZINC12323863 inhibits peak sodium current non-selectively in both Naᵥ1.1 and Naᵥ1.5 by ˜80% (FIG. 10).

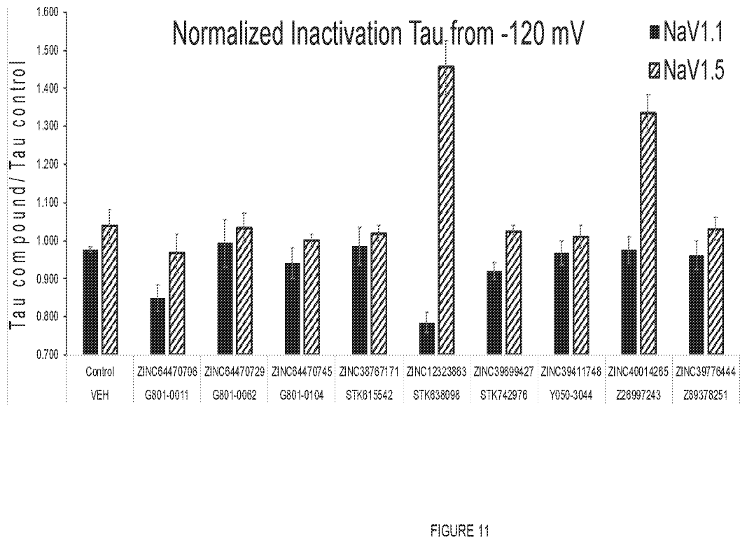

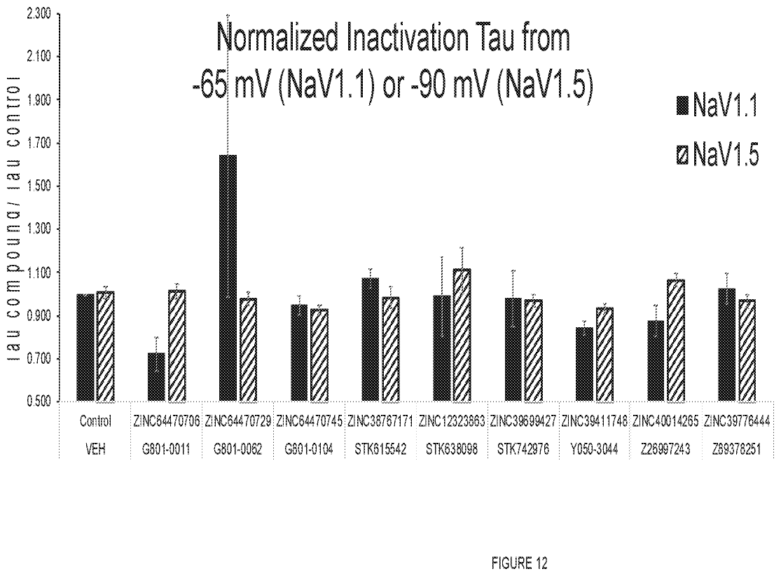

Effects of the nine compounds on fast inactivation time constant was measured by normalizing the compound time constant to control. ZINC12323863 and ZINC40014265 selectively decelerate fast inactivation onset kinetics by 1.5-fold compared to control and the other compounds at 50 μM in Naᵥ1.5 compared to Naᵥ1.1 (FIG. 11). This effect is only observed after preconditioning the channels to a potential of −120 mV. However, the deceleration in fast inactivation is not observed when preconditioning channels to mid-voltage potentials of Naᵥ1.1 and Naᵥ1.5 for 10 s (FIG. 12) suggesting that these compounds elicit their mechanism of action by binding to the resting state of the channel. The ability of these compounds to decelerate fast inactivation may be impeded by accumulation of channels in slow inactivation by the double-pulse protocol. Furthermore, ZINC12323863 accelerates kinetics of fast inactivation in Naᵥ1.1 compared to Naᵥ1.5.

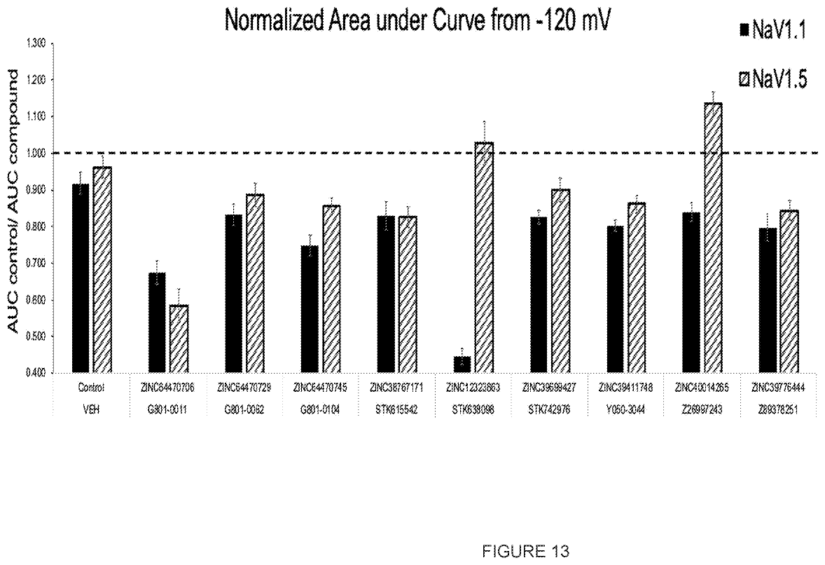

The area-under-curve (AUC) of peak I_{Na} measured following a −120 mV holding potential was analyzed by comparing the AUC of compound to compared (FIG. 13). ZINC40014265 shows a selective increase in AUC in Naᵥ1.5 compared to Naᵥ1.1. This suggests an increase in the charge that is mediated by voltage-sensors resulting in an overall increase in the channel's potentiation by enhancing channel activation. In contract, ZINC12323863 has a marked lower AUC in Naᵥ1.1 compared to Naᵥ1.5 (and other compounds) since its non-selective inhibition in Naᵥ1.1 is not counteracted by a deceleration in fast inactivation kinetics as in Naᵥ1.5. Thus, ZINC12323863 can be classified as a compound with mild potentiation effects in Naᵥ1.5.,

The results indicate that ZINC40014265 and ZINC12323863 selectively target the biophysical underpinnings of loss-of-function by decelerating inactivation in cardiac sodium channels (Naᵥ1.5) compared to neuronal sodium channels (Naᵥ1.1). ZINC40014265 did not inhibit peak sodium current.

All citations are hereby incorporated by reference.

The present invention has been described with regard to one or more embodiments. However, it will be apparent to persons skilled in the art that a number of variations and modifications can be made without departing from the scope of the invention as defined in the claims.

### REFERENCES

1. Zipes, D. P. & Wellens, H. J. J. Sudden Cardiac Death. Circulation 98, 2334-2351 (1998).
2. Laslett, L. J. et al. The Worldwide Environment of Cardiovascular Disease: Prevalence, Diagnosis, Therapy, and Policy Issues. J. Am. Coll. Cardiol. 60, S1-S49 (2012).
3. Yang, N. & Horn, R. Evidence for voltage-dependent S4 movement in sodium channels. Neuron 15, 213-218 (1995).
4. Ulbricht, W. Sodium Channel Inactivation: Molecular Determinants and Modulation. Physiol. Rev. 85, 1271-1301 (2005).
5. Yarov-Yarovoy, V. et al. Structural basis for gating charge movement in the voltage sensor of a sodium channel. Proc. Natl. Acad. Sci. 109, E93-E102 (2012).
6. Armstrong, C. M. & Bezanilla, F. Inactivation of the sodium channel. II. Gating current experiments. J. Gen. Physiol. 70, 567-590 (1977).
7. Ragsdale, D. S., McPhee, J. C., Scheuer, T. & Catterall, W. A. Common molecular determinants of local anesthetic, antiarrhythmic, and anticonvulsant block of voltage-gated Na+ channels. Proc. Natl. Acad. Sci. U.S.A 93, 9270-9275 (1996).
8. Capes, D. L., Goldschen-Ohm, M. P., Arcisio-Miranda, M., Bezanilla, F. & Chanda, B. Domain IV voltage-sensor movement is both sufficient and rate limiting for fast inactivation in sodium channels. J. Gen. Physiol. 142, 101-112 (2013).
9. Maltsev, V. & Undrovinas, A. A multi-modal composition of the late Na+ current in human ventricular cardiomyocytes. Cardiovasc. Res. 69, 116-127 (2006).
10. Richmond, J. E., Featherstone, D. E., Hartmann, H. A. & Ruben, P. C. Slow inactivation in human cardiac sodium channels. Biophys. J. 74, 2945-2952 (1998).
11. Vilin, Y. Y., Fujimoto, E. & Ruben, P. C. A single residue differentiates between human cardiac and skeletal muscle Na+ channel slow inactivation. Biophys. J. 80, 2221-2230 (2001).
12. Payandeh, J., Gamal El-Din, T. M., Scheuer, T., Zheng, N. & Catterall, W. A. Crystal structure of a voltage-gated sodium channel in two potentially inactivated states. Nature (2012) doi:10.1038/nature11077.
13. Qu, Y., Rogers, J. C., Tanada, T. N., Catterall, W. A. & Scheuer, T. Phosphorylation of S1505 in the cardiac Na+ channel inactivation gate is required for modulation by protein kinase C. J. Gen. Physiol. 108, 375-379 (1996).
14. Meadows, L. & Isom, L. Sodium channels as macromolecular complexes: Implications for inherited arrhythmia syndromes. Cardiovasc. Res. 67, 448-458 (2005).
15. Herren, A. W., Bers, D. M. & Grandi, E. Post-translational modifications of the cardiac Na channel: contribution of CaMKII-dependent phosphorylation to acquired arrhythmias. Am. J. Physiol. Heart Circ. Physiol. 305, H431-445 (2013).
16. Song, L. S. et al. beta-Adrenergic stimulation synchronizes intracellular Ca(2+) release during excitation-contraction coupling in cardiac myocytes. Circ. Res. 88, 794-80.1 (2001).
17. Baartscheer, A., Schumacher, C. A., Belterman, C. N. W., Coronel, R. & Fiolet, J. W. T. SR calcium handling and calcium after-transients in a rabbit model of heart failure. Cardiovasc. Res. 58, 99-108 (2003).
18. Wagner, S. et al. Ca2+/calmodulin-dependent protein kinase II regulates cardiac Na+ channels. J. Clin. Invest. 116, 3127-3138 (2006).
19. Chen, Y., Yu, F. H., Surmeier, D. J., Scheuer, T. & Catterall, W. A. Neuromodulation of Na+ Channel Slow Inactivation via cAMP-Dependent Protein Kinase and Protein Kinase C. Neuron 49, 409-420 (2006).
20. Dumaine, R. et al. Multiple mechanisms of Na+ channel-linked long-QT syndrome. Circ. Res. 78, 916-924 (1996).
21. Fredj, S., Lindegger, N., Sampson, K. J., Carmeliet, P. & Kass, R. S. Altered Na+ channels promote pause-induced spontaneous diastolic activity in long QT syndrome type 3 myocytes. Circ. Res. 99, 1225-1232 (2006).
22. Makita, N. et al. The E1784K mutation in SCN5A is associated with mixed clinical phenotype of type 3 long QT syndrome. J. Clin. Invest. (2008) doi:10.1172/JCI34057.
23. Amin, A. S., Asghari-Roodsari, A. & Tan, H. L. Cardiac sodium channelopathies. Pflüg. Arch.-Eur. J. Physiol. 460, 223-237 (2010).
24. Amin, A. S., Tan, H. L. & Wilde, A. A. M. Cardiac ion channels in health and disease. Heart Rhythm 7, 117-126 (2010).
25. Kaczmarski, J. A. & Corry, B. Investigating the size and dynamics of voltage-gated sodium channel fenestrations: A molecular dynamics study. Channels 8, 264-277 (2014).
26. Montini, G., Booker, J., Sula, A. & Wallace, B. A. Comparisons of voltage-gated sodium channel structures with open and closed gates and implications for state-dependent drug design. Biochem. Soc. Trans. 46, 1567-1575 (2018).
27. Gamal El-Din, T. M., Lenaeus, M. J., Zheng, N. & Catterall, W. A. Fenestrations control resting-state block of a voltage-gated sodium channel. Proc. Natl. Acad. Sci. 115, 13111-13116 (2018).
28. Li, H. L., Galue, A., Meadows, L. & Ragsdale, D. S. A molecular basis for the different local anesthetic affinities of resting versus open and inactivated states of the sodium channel. Mol. Pharmacol. 55, 134-141 (1999).
29. Payandeh, J., Scheuer, T., Zheng, N. & Catterall, W. A. The crystal structure of a voltage-gated sodium channel. Nature 475, 353-358 (2011).
30. Boiteux, C. et al. Local anesthetic and antiepileptic drug access and binding to a bacterial voltage-gated sodium channel. Proc. Natl. Acad. Sci. U.S.A 111, 13057-13062 (2014).
31. Shen, H. et al. Structure of a eukaryotic voltage-gated sodium channel at near-atomic resolution. Science 355, eaal4326 (2017).
32. Jiang, D. et al. Structure of the Cardiac Sodium Channel. Cell 180, 122-134.e10 (2020).
33. Nguyen, P. T., DeMarco, K. R., Vorobyov, I., Clancy, C. E. & Yarov-Yarovoy, V. Structural basis for antiarrhythmic drug interactions with the human cardiac sodium channel. Proc. Natl. Acad. Sci. U.S.A 116, 2945-2954 (2019).
34. Balser, J. R., Nuss, H. B., Romashko, D. N., Marban, E. & Tomaselli, G. F. Functional consequences of lidocaine binding to slow-inactivated sodium channels. J. Gen. Physiol. 107, 643-658 (1996).
35. Sokolov, S., Peters, C. H., Rajamani, S. & Ruben, P. C. Proton-dependent inhibition of the cardiac sodium channel Naᵥ1.5 by ranolazine. Front. Pharmacol. 4, (2013).
36. Bordoli, L. et al. Protein structure homology modeling using SWISS-MODEL workspace. Nat. Protoc. 4, 1-13 (2008).
37. Fredj, S., Sampson, K. J., Liu, H. & Kass, R. S. Molecular basis of ranolazine block of LQT-3 mutant sodium channels: evidence for site of action. Br. J. Pharmacol. 148, 16-24 (2006).
38. Huang, H., Priori, S. G., Napolitano, C., O'Leary, M. E. & Chahine, M. Y1767C, a novel SCN5A mutation, induces a persistent Na+ current and potentiates ranolazine inhibition of Naᵥ1.5 channels. AJP Heart Circ. Physiol. 300, H288-H299 (2011).
39. Morris, G. M. et al. AutoDock4 and AutoDockTools4: Automated docking with selective receptor flexibility. J. Comput. Chem. 30, 2785-2791 (2009).

1. A method of inhibiting the inactivation of a Naᵥ1.5 voltage-gated sodium channel comprising contacting the Naᵥ1.5 voltage-gated sodium channel with a compound according to Formula I: 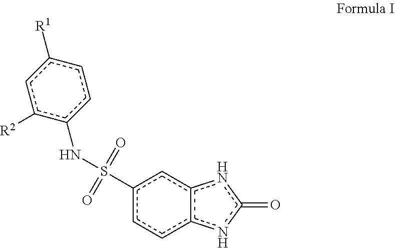
wherein R¹ is halo, and
R² is alkyl, alkenyl or alkynyl;
or Formula II: 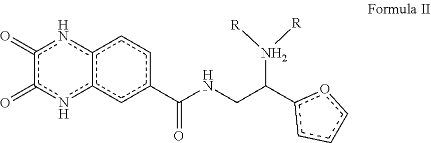
wherein R is each independently alkyl, alkenyl or alkynyl.

2. The method of claim 1 wherein the inactivation is slow inactivation, fast inactivation, or a combination thereof.

3. The method of claim 2 wherein the inactivation is slow inactivation and the compound is a compound according to Formula I.

4. The method of claim 2 wherein the inactivation is fast inactivation and the compound is a compound according to Formula II.

5. The method of claim 1 wherein the Naᵥ1.5 voltage-gated sodium channel is in an inactivated state or a closed state.

6. The method of claim 5 wherein the Naᵥ1.5 voltage-gated sodium channel is in an inactivated state and the compound is a compound according to Formula I.

7. The method of claim 5 wherein the Naᵥ1.5 voltage-gated sodium channel is in a closed state and the compound is a compound according to Formula II.

8. The method of claim 1 wherein the compound binds the Naᵥ1.5 voltage-gated sodium channel.

9. The method of claim 8 wherein the compound binds within a fenestration of the Naᵥ1.5 voltage-gated sodium channel.

10. A method of treating a cardiovascular disease comprising administering a compound that inhibits the inactivation of a Naᵥ1.5 voltage-gated sodium channel to a subject in need thereof or comprising inhibiting the inactivation of a Naᵥ1.5 voltage-gated sodium channel in a subject in need thereof.

11. The method of claim 10 wherein the cardiovascular disease is Brugada Syndrome, cardiac arrhythmia disorders, progressive cardiac conduction disorder (PCCD), sick sinus syndrome, progressive familial heart block, atrial fibrillation, sudden infant death syndrome, dilated cardiomyopathy, myocardial ischemia/infarction or heart failure.

12. The method of claim 10 wherein the subject is a human.

13. The method of claim 10 wherein the compound is a compound according to Formula I: 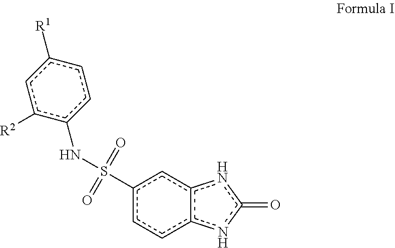
wherein R¹ is halo, and
R² is alkyl, alkenyl or alkynyl;
or Formula II: 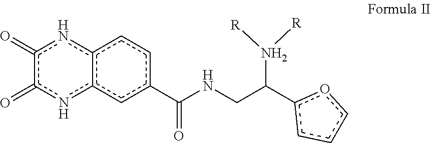
wherein R is each independently alkyl, alkenyl or alkynyl.

14. The method of claim 1 wherein the compound is selected from one or more of the compounds set forth in Table 2.

15. (canceled)

16. A pharmaceutical composition comprising a compound according to Formula I: 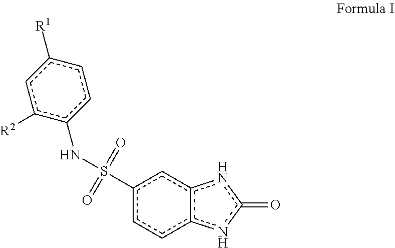
wherein R¹ is halo, and
R² is alkyl, alkenyl or alkynyl;
or Formula II: 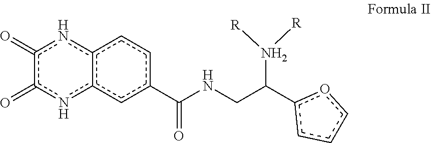
wherein R is each independently alkyl, alkenyl or alkynyl, in combination with a pharmaceutically acceptable carrier.

17. The pharmaceutical composition of claim 16 wherein the compound is selected from one or more of the compounds set forth in Table 2.

18. The method of claim 10 wherein the compound is selected from one or more of the compounds set forth in Table 2.
